(19) United States
(12) Patent Application Publication
(10) Pub. No.: US 2013/0197015 A1
(43) Pub. Date: Aug. 1, 2013

(54) NOVEL METHOD TO IMPROVE THE SAFETY AND EFFICACY OF CAFFEINE

(71) Applicant: Pondera Biotechnologies, LLC, Pownal, ME (US)

(72) Inventors: Steven CRAIN, State College, PA (US); William E. Crain, Pownal, ME (US); Stanley M. Crain, State College, PA (US); Michael Crain, Essex, CT (US)

(73) Assignee: Pondera Biotechnologies, LLC, Pownal, ME (US)

(21) Appl. No.: 13/830,895

(22) Filed: Mar. 14, 2013

Related U.S. Application Data
(63) Continuation-in-part of application No. 13/764,543, filed on Feb. 11, 2013.
Application No. 13/764,543 is a continuation of application No. 13/460,467, filed on Apr. 30, 2012, now Pat. No. 8,372,414.
Application No. 13/460,467 is a continuation of application No. 12/973,839, filed on Dec. 20, 2010, now Pat. No. 8,202,525.
(60) Provisional application No. 61/289,293, filed on Dec. 22, 2009.
(60) Provisional application No. 61/309,766, filed on Mar. 2, 2010.
(60) Provisional application No. 61/322,665, filed on Apr. 9, 2010.
(60) Provisional application No. 61/323,465, filed on Apr. 13, 2010.
(60) Provisional application No. 61/330,631, filed on May 3, 2010.
(60) Provisional application No. 61/351,653, filed on Jun. 4, 2010.
(60) Provisional application No. 61/375,463, filed on Aug. 20, 2010.
(60) Provisional application No. 61/385,873, filed on Sep. 23, 2010.
(60) Provisional application No. 61/386,952, filed on Sep. 27, 2010.

(51) Int. Cl. A61K 31/522; A61K 45/06; A61K 31/198

(52) U.S. Cl. CPC A61K 31/522; A61K 31/198; A61K 45/06
USPC 514/263.31

(57) ABSTRACT

The present invention relates to methods and compositions for reducing side effects associated with caffeine consumption. The methods comprise administering a Receptor Switcher, such as N-acetyl cysteine, in combination with caffeine.

### CROSS-REFERENCE TO RELATED APPLICATIONS

This application is a continuation-in-part of U.S. patent application Ser. No. 13/764,543, filed on Feb. 11, 2013, which is a continuation of U.S. Patent application Ser. No. 13/460,467, filed on Apr. 30, 2012, now U.S. Pat. No. 8,372,414, which is a continuation of U.S. patent application Ser. No. 12/973,839, filed Dec. 20, 2012, now U.S. Pat. No. 8,202,525, which claims priority of U.S. Patent Application No. 61/289,293, filed on Dec. 22, 2009, U.S. Patent Application No. 61/309,766, filed on Mar. 2, 2010, U.S. Patent Application No. 61/322,665, filed on Apr. 9, 2010, U.S. Patent Application No. 61/323,465, filed on Apr. 13, 2010, U.S. Patent Application No. 61/330,631, filed on May 3, 2010, U.S. Patent Application No. 61/351,653, filed on Jun. 4, 2010, U.S. Patent Application No. 61/375,463, filed on Aug. 20, 2010, U.S. Patent Application No. 61/385,873, filed on Sep. 23, 2010, and U.S. Patent Application No. 61/386,952, filed on Sep. 27, 2010. The contents of these applications are incorporated by reference in their entirety.

### FIELD OF INVENTION

The present invention relates to methods and compositions for improving the safety and efficacy of caffeine.

### BACKGROUND OF THE PRIOR ART

### I. Background Regarding Caffeine

Caffeine (1,3,4-trimethylxanthine), a phosphodiesterase (PDE) Inhibitor, is a natural alkaloid present in the leaves, fruits, and seeds of various plants (e.g., coffee, tea, guarana), and can also be synthesized. Caffeine is the most frequently ingested drug in the world since it is present in foods and drinks (coffee, tea, soft drinks, chocolate), dietary supplements, sports and energy drinks, powders, and pills, dietary supplements and over-the-counter and prescription medications. As a result, caffeine is consumed by millions of Americans daily. “Caffeine is consumed by 80-90% of Americans on a daily basis, making it one of the most commonly used drugs in our society” (www.stress.about.com/cs/substanceabuse/a/aa070202.htm).

Unfortunately, caffeine has also been linked to a wide variety of health-related problems due to its biophysiological effects on the brain and body. Caffeine has been shown to have adverse side effects such as increased stress and anxiety, agitation, restlessness, insomnia, irritability and anger, and cravings. For instance, a study by researchers at Duke University has linked caffeine consumption to increased levels of stress in the body. According to Dr. Lane (2002), “the effects of coffee drinking are long-lasting and exaggerate the stress response both in terms of the body's physiological response in blood pressure elevations and stress hormonal levels, but is also magnifies a person's perception of stress.” Lane, J. D. (2002) News Release, Duke University Medical Center, Caffeine's Effects are Long-Lasting and Compound Stress, http://www.dukemednews.org/news/article.php?id=5687 (full article appears in the July/August 2002 issue of Psychosomatic Medicine.) Another study at Ohio University by Drs. France and Ditto (1992) found that “questionnaires administered during baseline periods to assess psychological responses to stress and caffeine revealed a potentiation of anxiety and anger responses to stress”. France, C. & Ditto, B. (1992), Cardiovascular Responses to the Combination of Caffeine and Mental Arithmetic, Cold Pressor, and Static Exercise Stressors, Psychophysiology, 29: 272-82. Many studies have shown long-lasting noxious side effects of caffeine consumption. While generally dose and duration dependent, there is considerable evidence of individual variability. Some individuals experience serious side effects with even small amounts of caffeine, such as the amount in a cup of coffee or even a cup of tea.

There is also significant evidence that people can become dependent on caffeine and experience withdrawal symptoms when use is discontinued (Hartley et al, 2004). Hartley, T. et al (2004), Caffeine and Stress: Implications for Risk, Assessment, and Management of Hypertension, Am. J. of Cardiology, 93: 1022-6. Suddenly stopping consumption of caffeine may result in withdrawal symptoms including headaches, fatigue, depression, irritability, insomnia, anxiety, concentration problems, nervousness, and tension and other signs of drug dependence and addiction. For instance, a study at Johns Hopkins by Drs. Griffiths and Juliano (2004) documented the serious problems of caffeine tolerance, dependence, withdrawal, and addiction. Griffiths, R. & Juliano, L. (2004), News release, Johns Hopkins University Department of Neuroscience, Caffeine Withdrawal Recognized as a Disorder (September 2004). (Full study published in the October 2004 issue of the Journal of Psychopharmacology). Due to withdrawal symptoms, consumers maintain frequent daily consumption of caffeine to avoid withdrawal, resulting in an addiction to caffeine. Another significant factor is that many people become tolerant to caffeine, requiring higher and higher doses to experience benefits and to avoid withdrawal symptoms. As a result, many people find themselves taking higher and higher doses of caffeine, causing greater side effects over time.

The problems with caffeine consumption have become extremely serious with the advent of our society's extensive use of highly caffeinated products, including coffee and energy drinks. In fact, the concerns over the serious side effects of this high level of caffeine consumption has recently led the FDA to begin an inquiry into the noxious and even fatal consequences (http://www.fda.gov/Food/NewsEvents/ucm328536.htm) of caffeinated products.

Unfortunately, there are currently no methods available to reduce the noxious side effects of caffeine, other than abstinence. As the most widely used drug in our society, there is a critical need to discover safe and effective methods to reduce the deleterious effects of caffeine, including anxiety, restlessness, agitation, insomnia, anger, tension, and cravings, as well as the development of tolerance, dependence, withdrawal, and addiction. The present invention satisfies this need.

### II. Background Regarding Related Applications

Examples of prior art directed to the treatment of pain, IBS, and other Distress Dysfunctions include US Patent Publication No. 20020198227 for “Method for curbing dietary craving” (describing administration of low dose naltrexone); US Patent Publication No. 20030191147 for “Opioid antagonist compositions and dosage forms”; US Patent Publication No. 20030211157 for “Semi-sol delivery blend for water soluble molecules”; US Patent Publication No. 20040072864 for “Method and composition for treatment of irritable bowel disease”; US Patent Publication No. 20060009478 for “Methods for the treatment of back pain” (does not describe the use of ultra low does naltrexone in combination with a non-opioid analgesic); US Patent Publication No. 20060069086 for “Methods for regulating neurotransmitter systems by inducing counteradaptations” (does not describe ultra low does naltrexone or co-treatment); US Patent Publication No. 20070099947 for “Methods and compositions for the treatment of brain reward system disorders by combination therapy” (does not describe ultra low does naltrexone); US Patent Publication No. 20080045610 for “Methods for regulating neurotransmitter systems by inducing counteradaptations”; US Patent Publication No. 20080207601 for “Methods of and Compositions For the Prevention of Anxiety, Substance Abuse, and Dependence” (cotreatment agents are steroids); US Patent Publication No. 20080255097 for “Methods for the Treatment of Substance Abuse and Dependence”; US Patent Publication No. 20100144645 for “Compositions and Methods for Enhancing Analgesic Potency of Covalently Bound-Compounds . . . ”; US Patent Publication No. 20100168119 for “Compositions and Methods for Minimizing or Reducing Agonist-Induced Desensitization”; U.S. Pat. Nos. 6,458,795, 6,664,270 and 6,818,656, all for “Method and composition for treatment of irritable bowel disease”; U.S. Pat. No. 6,972,291 for “Method for reducing food intake” (describing administration of low dose (not ultra low does) naltrexone); WO 2007/056300 for “Methods and Compositions for the Treatment of Brain Reward System Disorders by Combination Therapy”; WO 2000/067739 for “Opioid Antagonists Containing Compositions for Enhancing Analgesic Potency of Tramadol and Attenuating its Adverse Side Effects”; WO 2006/034343 for “Methods for regulating neurotransmitter systems by inducing counteradaptations”; WO 2006/110557 for “Methods for the Treatment of Substance Abuse and Dependence”; WO 2007/100775 for “Methods for Regulating Neurotransmitter Systems by Inducing Counteradaptations”; WO 2007/120864 for “Compositions and Methods for Enhancing Analgesic Potency of Covalently Bound Compounds . . . ”; WO 2008/094571 for “Methods for Treating Acute and Subchronic Pain”; WO 2009/017625 for “Treatment of Depression, Psychosis, and Anxiety”; and WO 2010/053835 for “Compositions and Methods for Minimizing or Reversing Agonist-Induced Desensitization.” However, there is no prior art that teaches the critical importance of understanding the role of protracted excitatory opioid (and related) receptor signaling, together with diminished opioid (and related) neurotransmitters, in Distress Dysfunctions, and its clinical implication for the use of Receptor Balancers combined with Endorphin Enhancers and/or Synergistic Enhancers and/or Exogenous Opioids for safe and effective treatment.

### SUMMARY OF THE INVENTION

This present invention is directed to the discovery that the addition of a Receptor Switcher, as defined below, to caffeine, a PDE inhibitor, (1) reduces the side effects that would occur when the PDE inhibitor is administered alone, and (2) improves the therapeutic efficacy of the PDE inhibitor (caffeine) when administered alone. The compositions and methods of the invention result in the surprising and dramatic synergistic reduction of side effects associated with caffeine consumption.

This invention is directed to compositions and methods which improve the safety and efficacy of caffeine. The methods comprise co-administration of caffeine with a Receptor Switcher, as defined herein. For example, the Receptor Switcher can be selected from the group consisting of agents that selectively block and/or inhibit opioid receptor excitatory signaling, ultra-low-dose and very-low-dose opioid antagonists, ultra-low-dose and very-low-dose naltrexone, naloxone, diprenorphine, nalmefene, and norbinaltorphimine, agents that inhibit synthesis or activity of GM1 ganglioside, neuraminidase inhibitors, agents that increase sulfates in the body, methylsulfonylmethane (MSM), magnesium sulfate, sodium sulfate, chondroitin sulfate, n-acetyl-cysteine (NAC), zanamivir, laninamivir, peramivir, oseltamivir, scutellaria, and 5,7,4′-trihydroxy-8-methoxyflavone.

In yet another embodiment of the invention, the subject has previously been prescribed an ENDORPHIN ENHANCER, an EXOGENOUS OPIOID, a SYNERGISTIC ENHANCER, or any combination thereof.

In yet another embodiment of the invention, the RECEPTOR SWITCHER is a low dose opioid antagonist. For example, the RECEPTOR SWITCHER can be low dose naltrexone or naloxone. In another embodiment, naltrexone as a RECEPTOR SWITCHER can be administered: (a) in the ultra low dose amount of about 125 micrograms or less; (b) in the very-low-dose range of about 125—about 500 micrograms; or (c) in the low-dose range of about 500—about 5000 micrograms. In another embodiment, naloxone as a RECEPTOR SWITCHER can be administered: (a) at an ultra low dosing of about 0.25 μg·kg⁻¹·h⁻¹; (b) at about 400 micrograms naloxone in 1000 ml crystalloid given in 24 h to a patient weighing 70 kg; (c) at a-low-dosing of about 1.0 μg·kg⁻¹·h⁻¹; or (d) at about 0.01 μg·kg⁻¹·h⁻¹ to about 5 μg·kg⁻¹·h⁻¹.

In yet another embodiment of the invention, encompassed is a method wherein the RECEPTOR SWITCHER is selected from the group consisting of a neuramindase inhibitor, n-acetyl-cysteine (NAC), magnesium sulfate, and methylsulfonylmethane (MSM).

Any pharmaceutically acceptable administration method can be used for the invention. For example, the mode of administration can be selected from the group consisting of oral, pulmonary, nasal, sublingual, parenteral, transdermal, topical, and suppository. Moreover, the composition of the invention can be in any pharmaceutically acceptable dosage form. For example, the pharmaceutical formulation used in the methods of the invention can be delivered in a pharmaceutically-acceptable carrier that is rapid release, immediate-release, slow-release, controlled-release, delayed-released, and combination controlled and immediate release, including nano-encapsulation formulations, as well as any and all technologies that maximize therapeutic effectiveness, such as extended and slow release of the pharmaceutical formulation. In addition, the pharmaceutical formulation used in the methods of the invention can be delivered in an abuse-resistant delivery system.

In another embodiment, the side effects associated with caffeine consumption and treated or minimized using the compositions and methods of the invention include, but are not limited to: (1) Mood Disorders; (2) Eating Disorders; (3) Gastrointestinal Disorders; (4) Pre-Menstrual Syndrome (PMS) and other hormonally-related distress signs and symptoms; (5) Sleep Disorders; (6) Caffeine-Related Disorders; (7) Psychotic Disorders; (8) Behavioral addictions, compulsions, and dysfunctions; and (9) Emotional and physical malaise, distress, discomfort, pain, restlessness, irritability, worries, cravings, compulsions, obsessions, agitation, addictions, and other related complaints and signs of protracted negative hedonic tone that may, or may not, be part of a traditional medical or psychiatric disorder. In particular, noted side effects of caffeine include but are not limited to increased stress and anxiety, agitation, restlessness, insomnia, irritability and anger, cravings, stomach pain (tenderness, bloating), constipation, unusual weakness, seizure (convulsions), twitching or uncontrolled muscle movements, fever, fast or slow heart rate, loss of appetite, anxiety, restlessness, depression, aggravation of premenstrual syndrome (PMS), fibrocystic breast disease, psychiatric side effects (confusion and psychotic symptoms), increased blood pressure, decrease in insulin sensitivity, hypoglycemia, and hyperglycemia.

Examples of Mood Disorders include, but are not limited to, Depressive Disorders, Dysthymic Disorder, Bipolar I Disorder, Bipolar II Disorder, Bipolar Disorder NOS, Cyclothymic Disorder, Mood Disorders Related to Medical Conditions, Seasonal Affective Disorder, Mood Disorders NOS, as well as signs and symptoms of depressed mood, anhedonia, despair, anhedonia, hypomania, mania, and negative hedonic tone that are not classified as a Mood Disorder. Examples of Eating Disorders include, but are not limited to, Bulimia Nervosa, Anorexia Nervosa, Binge Eating, Eating Disorder NOS, as well as signs and symptoms of eating and appetite problems that are not classified as an Eating Disorder. Examples of Gastrointestinal Disorders include, but are not limited to, Irritable Bowel Syndrome (IBS) with Predominately Diarrhea, IBS with Predominately Constipation, and IBS Mixed Type, Crohn's Disease, as well as GI distress including, but not limited to, nausea, vomiting, diarrhea, constipation, and bloating. Examples of Sleep Disorder include, but are not limited to, Insomnia, Dyssomnias, Parasomnias as well as signs and symptoms of sleep problems that are not classified as a Sleep Disorder. Examples of a Caffeine-Related Disorder include, but are not limited to, Caffeine Dependence, Caffeine Addiction, Caffeine Abuse, Caffeine-Induced Disorders, Caffeine-Related Disorders NOS as well as caffeine-related problems that are not classified as a Caffeine-Related Disorder. Examples of a Behavioral addiction, compulsion, and/or dysfunction include, but are not limited to, sex, pornography, gambling, shopping, eating, drinking, smoking, computer use, and cleaning. An example of a Psychotic disorder is schizophrenia.

In another embodiment of the invention, encompassed are compositions useful in the methods of the invention. An exemplary composition useful in safely and effectively treating side effects associated with caffeine comprises: (a) at least one RECEPTOR SWITCHER; and (b) caffeine.

In one embodiment of the invention, encompassed is a composition wherein the RECEPTOR SWITCHER: (a) when co-administered with caffeine, reduces and/or resolves one or more undesirable side-effects or symptoms associated with caffeine consumption; and (b) can be selected from the group consisting of agents that selectively block and/or inhibit opioid receptor excitatory signaling, ultra-low-dose, very-low-dose, and low-dose opioid antagonists, ultra-low-dose, very-low-dose, and low-dose naltrexone, naloxone, diprenorphine, nalmefene, and norbinaltorphimine, agents that inhibit synthesis or activity of GM1 ganglioside, neuraminidase inhibitors, agents that increase sulfates in the body, methylsulfonylmethane (MSM), magnesium sulfate, sodium sulfate, chondroitin sulfate, n-acetyl-cysteine (NAC), zanamivir, oseltamivir, laninamivir, peramivir, scutellaria, and 5,7,4′-trihydroxy-8-methoxyflavone.

In one embodiment of the invention, encompassed is a composition wherein the RECEPTOR SWITCHER: (a) is an ultra-low-dose, very-low-dose, or low-dose opioid antagonist; and/or (b) is ultra-low-dose, very-low-dose, or low-dose naltrexone; and/or (c) is ultra-low-dose, very-low-dose, or low-dose naloxone. In such a composition, (a) the naltrexone can be administered in an ultra low dose range of about 125 micrograms or less; (b) the naltrexone can be administered in a very low dose range of about 125 to about 500 micrograms; (c) the naltrexone can be administered in the low dose range of about 500 to about 5000 micrograms; (d) the dosing of naloxone can be an ultra low dosing of about 0.25 μg·kg⁻¹·h⁻¹; (e) the dosing of naloxone can be about 400 micrograms naloxone in 1000 ml crystalloid given in 24 h to a patient weighing 70 kg; (f) the dosing of naloxone can be a very-low-dosing of about 1.0 μg·kg⁻¹·h⁻¹; and/or (g) the dosing range of naloxone can be 0.01 μg·kg⁻¹·h⁻¹ to 5 μg·kg⁻¹·h⁻¹.

In another embodiment of the invention, encompassed is a composition wherein the RECEPTOR SWITCHER is selected from the group consisting of a neuramindase inhibitor, n-acetyl-cysteine (NAC), scutellaria, 5,7,4′-trihydroxy-8-methoxyflavone, magnesium sulfate, sodium sulfate, and methylsulfonylmethane (MSM).

In another embodiment of the invention, encompassed is a composition comprising caffeine, and the RECEPTOR SWITCHER is ultra-low-dose, very-low-dose, and low-dose opioid antagonists, ultra-low-dose naltrexone or naloxone, very-low-dose naltrexone or naloxone, low-dose naltrexone or naloxone, neuraminidase inhibitors, n-acetyl-cysteine (NAC), magnesium sulfate, methylsulfonylmethane (MSM), or any combination thereof.

The compositions of the invention can be formulated into any pharmaceutically acceptable dosage forms. For example, the compositions of the invention can be formulated into a dosage form selected from the group consisting of rapid release, immediate-release, slow-release, sublingual, intravenous, controlled release, delayed-release, a combination of immediate and controlled release, nano-encapsulation formulations, and a tamper-resistant or abuse-resistant delivery system.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### DESCRIPTION OF THE FIGURES

FIG. 1: Shows the basic biochemical processes of Bimodal Endorphinergic Modulation of Pain and Hedonic Tone;

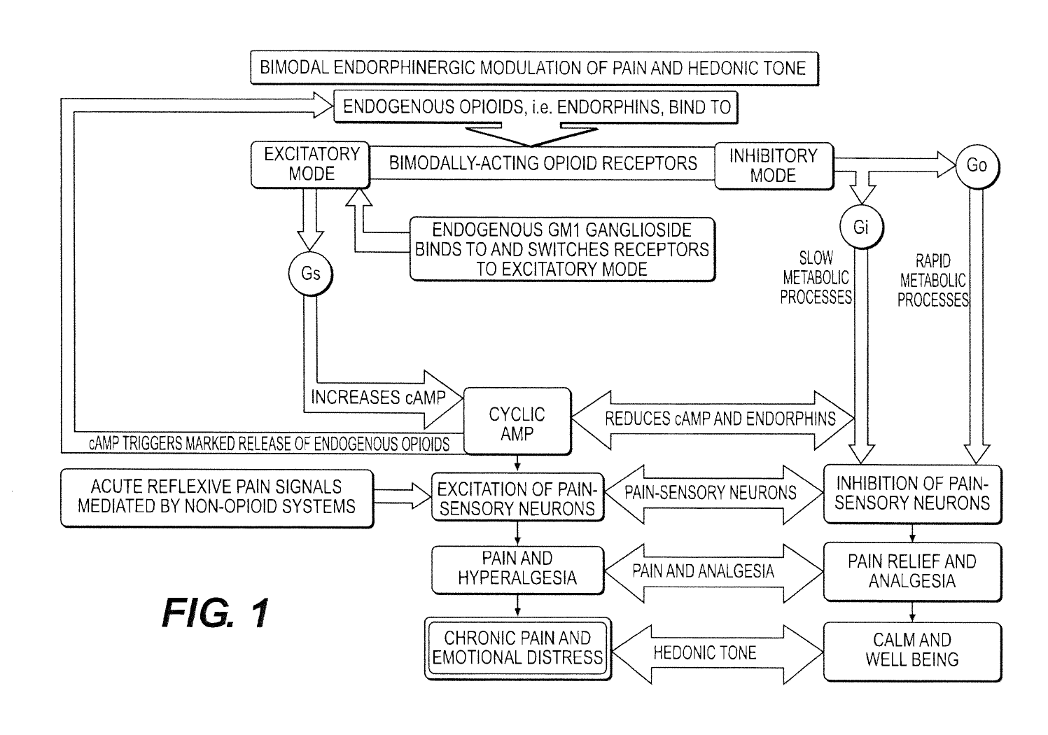

FIG. 2: Shows the transmission of normal pain and distress signals defined as Bimodal Endorphinergic Impact on Pain and Hedonic Tone (transmission of normal pain and distress signals);

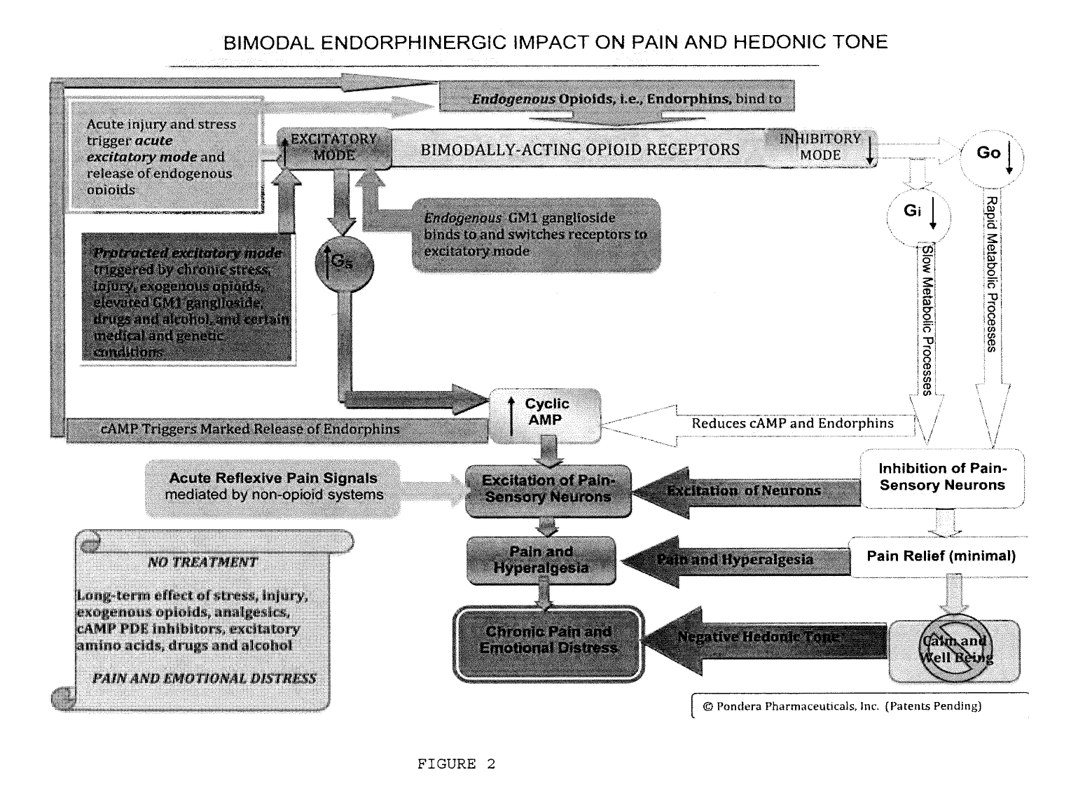

FIG. 3: Shows the acute impact of conventional drugs defined as Acute Unbalanced Endorphinergic Impact on Pain and Hedonic Tone;

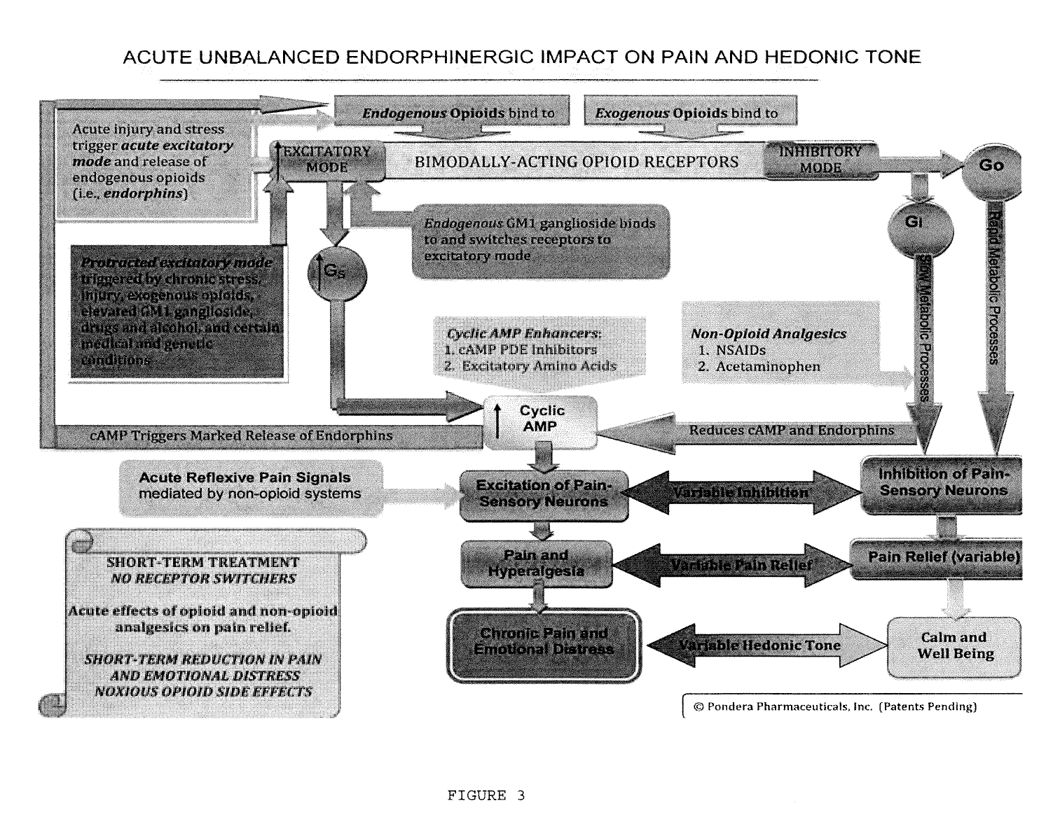

FIG. 4: Shows the long-term impact of conventional drugs defined as Chronic Unbalanced Endorphinergic Impact on Pain and Hedonic Tone; and

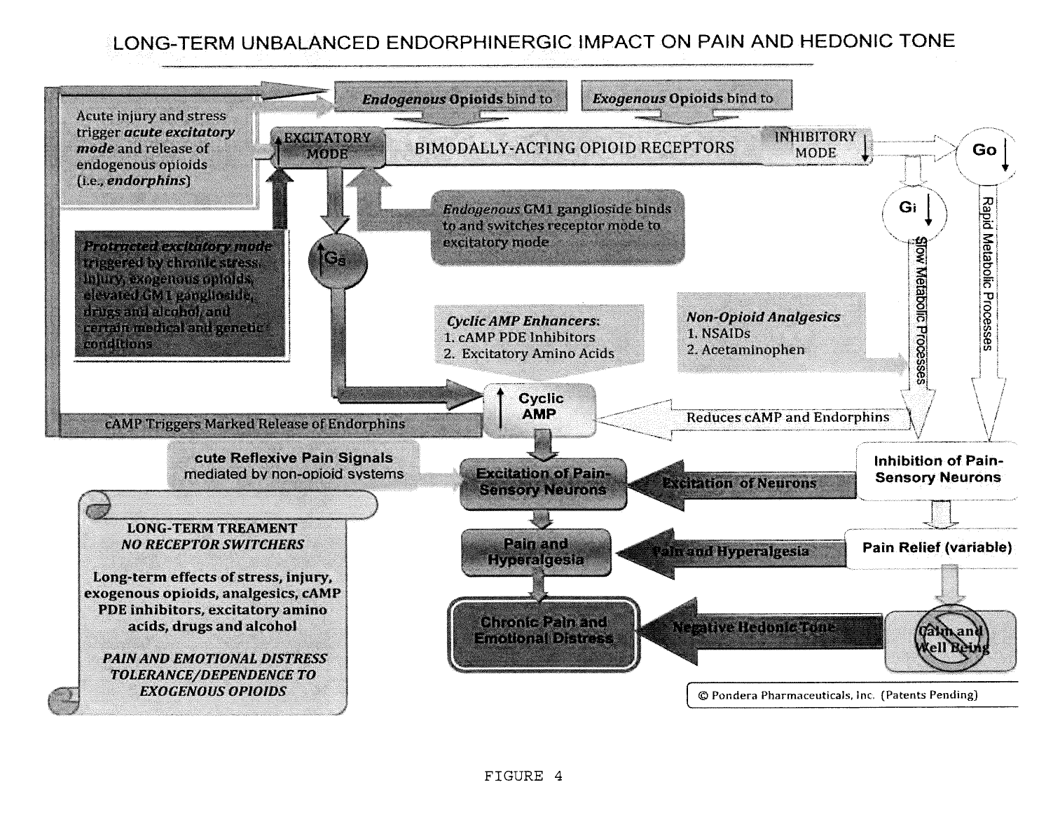

FIG. 5: Shows acute and long-term impact of novel balanced cotreatment formulations defined as Balanced Endorphinergic Impact on Pain and Hedonic Tone.

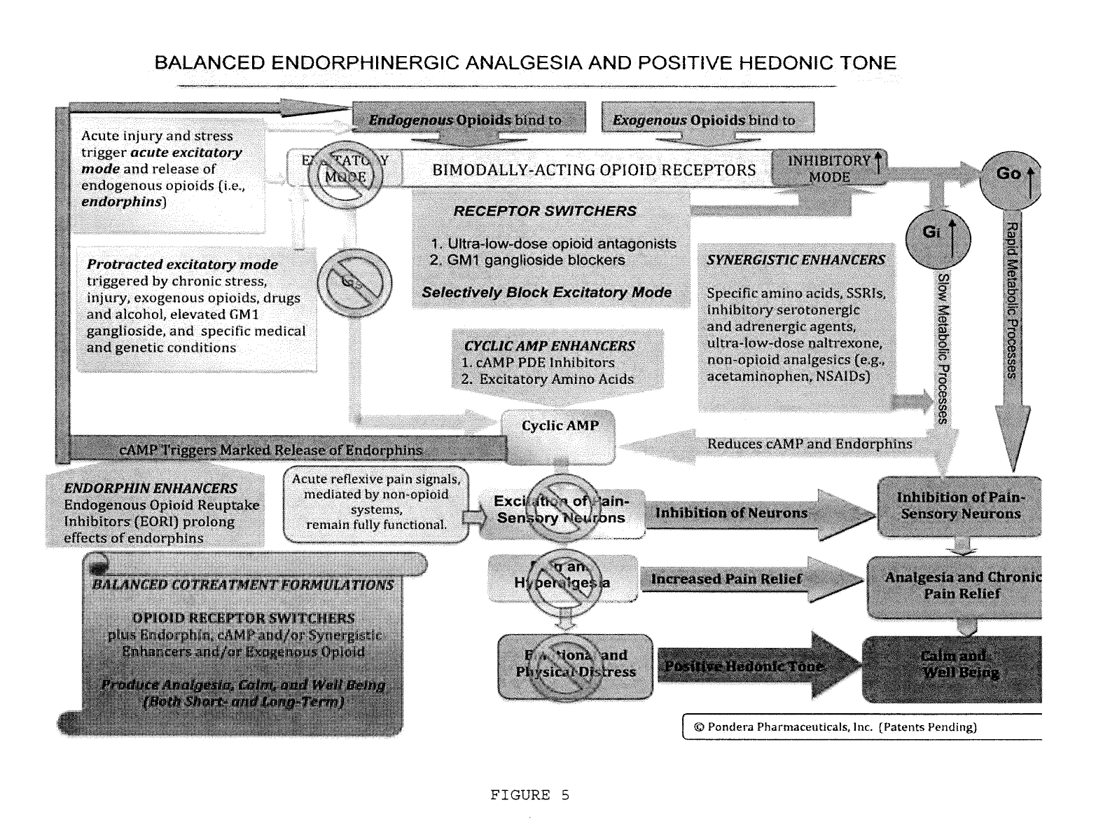

### DETAILED DESCRIPTION OF INVENTION

### I. Overview

Throughout the parent applications it is noted that one type of Distress Disorder was “unpleasant or deleterious side effects of CLASS II, III, or IV agents when such agents are administered alone (i.e., without co-administration with an CLASS I agent) which may, or may not, interfere with the potential therapeutic benefits of CLASS II, III, or IV agents, including, but not limited to PDE inhibitors” such as caffeine, which are classified as a CLASS II (Endorphin Enhancer) agent.

It was also noted that “when administered alone, Endorphin Enhancers . . . are more likely to produce [opioid receptor] excitatory signaling, thereby iatrogenically triggering symptoms of Distress Dysfunction, such as . . . anxiety and gastrointestinal symptoms, . . . . However, when co-administered with Receptor Switchers, Endorphin Enhancers trigger inhibitory signaling, leading to enhanced and prolonged analgesia and well being. Therefore, combining one or more Receptor Switchers, such as . . . N-Acetyl Cysteine (NAC) . . . with one or more Endorphin Enhancers, such as . . . caffeine” reduces and/or eliminates caffeine's typical side effects and even “creates a remarkable new generation of non-opioid pharmaceutical and nutraceutical formulations for the treatment of a wide variety of Distress Dysfunctions, by rebalancing the endogenous opioid system, restoring a basal homeostatic inhibitory mode together with normal levels of endorphins.”

While the remarkable discovery that the simple addition of a receptor switcher such as NAC to caffeine reverses the side effects of caffeine and enhances its benefits was articulated in the parent applications, the best dosing combinations and schedules were not clear at that time. It was not until the data from the case studies and pain and stress trials described herein were systematically analyzed, and integrated with subsequent studies, which were conducted regarding the diverse Distress Disorder conditions, that a surprising and unexpected pattern of results was revealed. These results suggested that to maximally reduce caffeine side effects and increase caffeine's benefits, three new parameters were required: (1) specific receptor switcher (i.e., NAC)/caffeine ratio when caffeine-containing compounds are consumed; (2) specific minimum and maximum daily consumption amounts; and (3) specific 24 hours consumption schedule. By carefully following these novel parameters, the typical side effects experienced when consuming caffeine, including emotional and physical distress, anxiety and fears, agitation and restlessness, pain sensitivity as well as tolerance, dependence, and addiction to caffeine, were most dramatically reduced. Furthermore, these novel parameters created combinations of a receptor switcher, such as NAC, and caffeine that provided maximal therapeutic benefits, which were dramatically greater and more comprehensive than either agent alone, including enhanced well being, calming energy, mental clarity, and bodily comfort.

The studies referred to in the parent applications found evidence of the frequent side effects reported by consumers of caffeine, even when ingesting relatively small doses of caffeine, which is consistent with prior research. However, through a series of unexpected findings, as documented herein, it was discovered that the remarkable reduction and/or elimination of these caffeine side effects is obtained by simply adding a Receptor Switcher, as defined below. In fact, the studies revealed a very surprising and unexpected benefit of co-administering a Receptor Switcher with caffeine. Not only were the side effects eliminated, but also the subjects experienced a therapeutic benefit from the combination of agents that was not produced by either agent alone. These benefits included a reduction in prior emotional and physical distress, anxiety, anger, cravings, and an increase in calm, comfort, and well-being. To be clear, these therapeutic benefits were not just the elimination of side effects of caffeine, but were improvements experienced regarding pre-existing emotional and physical distress. Therefore, these surprising and unexpected findings were not only remarkable; they were contrary to all teachings, which taught that caffeine produces anxiety and distress. No prior teaching has ever suggested that caffeine, in combination with a Receptor Switcher, such as NAC, would help reduce anxiety and distress. More than one Receptor Switcher can be used in the compositions and methods of the invention.

The continued studies, based on these earlier discoveries, focused on the use of a specific Receptor Switcher, NAC, in combination with a specific PDE inhibitor, caffeine. In one embodiment, described herein, are unexpected and novel findings that specific dose ratios, specific dose ranges, and specific dosing schedules are required to eliminate caffeine's side effects as well as maximize its therapeutic benefits. Specifically, the data analysis using reports from a total of 121 subjects found that caffeine consumption at all levels, when taken alone, led to at least some degree of side effect report from nearly 40% of all participants, including emotional and/or physical distress (see Example 14). The primary benefit reported by most subjects taking caffeine alone was increased energy. These patterns are typical of those reported in prior research. However, when a receptor switcher such as NAC was added, it was found that a dramatic decrease in noxious side effects and increase in overall well being (in addition to energy), were reported by most subjects (Example 14).

Moreover, a detailed analysis of the data revealed a remarkable and unexpected pattern, which depended on the ratio of receptor switcher (e.g., NAC) and caffeine, as well as dosing ranges and schedules (Example 14). A summary of these findings is reflected in Chart 1 below (Example 14). Specifically, it was discovered that to maximally reduce side effects typically experienced with caffeine, a minimum of approximately an equal dose of a receptor switcher such as NAC is necessary. Below this 1:1 level of receptor switcher (e.g., NAC) and caffeine, typical side effects of caffeine were reduced, but not nearly as effectively. Furthermore, it was discovered that the maximal reduction of side effects was generally seen when the ratio of receptor switcher (e.g., NAC) to caffeine was approximately 3:1 or higher. Ratios above this did not appear to significantly improve side effect reduction. In other words, higher doses of NAC beyond this ratio were effective, but not necessary. Therefore, regardless of the source of caffeine, it was unexpectedly discovered that the ratio of receptor switcher (e.g., NAC) to caffeine that maximally reduced caffeine side effects was approximately in the range of about 1:about 1 to about 3: about 1.

Thus, in one embodiment of the invention compositions and methods of use thereof are encompassed comprising at least one receptor switcher and caffeine in a ratio of about 1 to about 2, about 1 to about 1.9, about 1 to about 1.8, about 1 to about 1.7, about 1 to about 1.6, about 1 to about 1.5, about 1 to about 1.4, about 1 to about 1.3, about 1 to about 1.2, about 1 to about 1.1, about 1 to about 1, about 1.1 to about 1, about 1.2 to about 1, about 1.3 to about 1, about 1.4 to about 1, about 1.5 to about 1, about 1.6 to about 1, about 1.7 to about 1, about 1.8 to about 1, about 1.9 to about 1, about 2 to about 1, about 2.1 to about 1, about 2.2 to about 1, about 2.3 to about 1, about 2.4 to about 1, about 2.5 to about 1, about 2.6 to about 1, about 2.7 to about 1, about 2.8 to about 1, about 2.9 to about 1, about 3 to about 1, about 3.1 to about 1, about 3.2 to about 1, about 3.3 to about 1, about 3.4 to about 1, about 3.5 to about 1, about 3.6 to about 1, about 3.7 to about 1, about 3.8 to about 1, about 3.9 to about 1, about 4 to about 1, or above 4 to about 1.

No earlier teaching would have anticipated that the addition of a receptor switcher (e.g., NAC) to caffeine would reduce caffeine's side effect profile, nor would any earlier teaching have predicted this therapeutic dosing range.

A second discovery stemming from this analysis of the data from the continuing studies was that the therapeutic benefits of adding a receptor switcher (e.g., NAC) to caffeine were also maximized in this dosing range. Specifically, the combination of a receptor switcher (e.g., NAC) and caffeine, approximately between about 1: about 1 to about 3: about 1 dosing ratio, most effectively eliminated caffeine's side effects, including emotional and physical distress, fears and anxiety, agitation and restlessness, irritability and anger, and pain sensitivity, and produced a sense of well being, calming energy, bodily comfort, and mental clarity instead. Therefore, the remarkable discoveries, contrary to prior arts, revealed a novel method to add specific ratios of NAC to caffeine in order to conventional caffeine's anxiolytic characteristic to anxiety relief.

Third, while a receptor switcher such as NAC has remarkably few side effects, it was found that daily doses higher than 2400 mg tend to produce certain GI symptoms for some individuals. Therefore, the findings suggest that maximal caffeine side effect reduction and benefit enhancement can be achieved with an upper limit of 2400 mg of a receptor switcher such as NAC combined with 800 mg of caffeine (about 3: about 1 ratio), consumed over the course 24 hours. Doses of caffeine over 800 mg, even when combined with up to 2400 mg of NAC, tended to produce a return of certain caffeine side effects, since the ratio of the two agents was lower than the ideal about 3: about 1 ratio.

In one embodiment of the invention, the receptor switcher, such as NAC, has a daily dose of less than or equal to about 4000 mg, less than or equal to about 4500 mg, less than or equal to about 3000 mg, less than or equal to about 3900 mg, less than or equal to about 3800 mg, less than or equal to about 3700 mg, less than or equal to about 3600 mg, less than or equal to about 3500 mg, less than or equal to about 3400 mg, less than or equal to about 3300 mg, less than or equal to about 3200 mg, less than or equal to about 3100 mg, less than or equal to about 3000 mg, less than or equal to about 2900 mg, less than or equal to about 2800 mg, less than or equal to about 2700 mg, less than or equal to about 2600 mg, less than or equal to about 2500 mg, less than or equal to about 2400 mg, less than or equal to about 2300 mg, less than or equal to about 2200 mg, less than or equal to about 2100 mg, less than or equal to about 2000 mg, less than or equal to about 1900 mg, less than or equal to about 1800 mg, less than or equal to about 1700 mg, less than or equal to about 1600 mg, less than or equal to about 1500 mg, less than or equal to about 1400 mg, less than or equal to about 1300 mg, less than or equal to about 1200 mg, less than or equal to about 1100 mg, less than or equal to about 1000 mg, less than or equal to about 900 mg, less than or equal to about 800 mg, less than or equal to about 700 mg, less than or equal to about 600 mg, less than or equal to about 500 mg, less than or equal to about 400 mg, less than or equal to about 300 mg, less than or equal to about 200 mg, less than or equal to about 100 mg/day, less than or equal to about 50 mg, or less than or equal to about 25 mg/day.

Successful preliminary trials and case studies have been done using about 25-about 500 mg of caffeine daily, though higher or lower doses are likely to be effective for certain formulations and indications. In another embodiment of the invention, the daily consumption of caffeine is selected from the group consisting of less than or equal to about 1500 mg, less than or equal to about 1400 mg, less than or equal to about 1300 mg, less than or equal to about 1200 mg, less than or equal to about 1100 mg, less than or equal to about 1000 mg, less than or equal to about 900 mg, less than or equal to about 800 mg, less than or equal to about 700 mg, with 600 mg, less than or equal to about 500 mg, less than or equal to about 400 mg, less than or equal to about 300 mg, less than or equal to about 200 mg, less than or equal to about 190, less than or equal to about 180, less than or equal to about 170, less than or equal to about 160, less than or equal to about 150, less than or equal to about 140, less than or equal to about 130, less than or equal to about 120, less than or equal to about 110, less than or equal to about 100, less than or equal to about 90, less than or equal to about 80, less than or equal to about 70, less than or equal to about 60, less than or equal to about 50, less than or equal to about 40, less than or equal to about 30, less than or equal to about 20, less than or equal to about 10, less than or equal to about 5, or less than or equal to about 1 mg of caffeine.

Nevertheless, the findings make it clear that caffeine, at all doses, should be combined with a receptor switcher, such as NAC, to maintain relative safety and effectiveness. Any amount of a receptor switcher helps to reduce caffeine's side effects and enhance its benefits, but certain dose ratios appear to maximally produce these benefits.

Fourth, the data analysis revealed that a receptor switcher (e.g., NAC) should be consumed at approximately the same time as the caffeine; maximal caffeine side effect reduction and benefit enhancement were seen when a receptor switcher (e.g., NAC) and caffeine were consumed simultaneously. When caffeine was administered more than 30 min. prior to NAC, side effects were reported. In contrast, when NAC was administered up to four hours prior to the caffeine, side effects were minimized and benefits maximized. Therefore, combining NAC and caffeine in a single product, at the ratios described above, maximally reduces caffeine side effects and enhances caffeine's benefits. Alternatively, a product in which the two agents were consumed separately could be effective as long as they were both consumed at approximately the same time. If administered separately, evidence suggests that administration of the receptor switcher (e.g., NAC) prior to, or at the same time as the caffeine would be preferable to prevent caffeine's typical side effects.

In one embodiment of the invention, at least one receptor switcher is administered simultaneously as the caffeine. In another embodiment the receptor switcher is administered sequentially with the caffeine. In another embodiment, the receptor switcher is administered before the caffeine, including but not limited to up to about 24 hours before caffeine consumption. At least one receptor switcher can also be administered about 1, about 2, about 3, about 4, about 5, about 6, about 7, about 8, about 9, about 10, about 15, about 30, about 45, about 60, about 75, about 90, about 105, about 120, about 135, about 150, about 165, about 180, or about 200 min. before the caffeine. In another embodiment of the invention, at least one receptor switcher can be administered at any time within 24 hours prior to caffeine consumption. In another embodiment of the invention, at least one receptor switcher can be administered at any time within 24 hours following caffeine consumption to prevent longer-term side effects of caffeine.

Obviously, caffeine consumption as well as sensitivity varies considerably, depending on individual differences and preferences. Therefore, the findings strongly suggest that a receptor switcher (e.g., NAC) should be co-administered whenever caffeine is present in a consumable product, even when caffeine levels are low, since many individuals are sensitive to the noxious effects of caffeine, short and long-term, including tolerance and dependence as well as emotional and physical distress, even at very low levels.

This invention covers a wide variety of applications and forms of administration, including foods and beverages, pills and capsules, dietary supplements, over-the-counter drugs, prescription medications, injections and gels, and essentially any form in which caffeine can be consumed. It also covers products in which the receptor switcher (e.g., NAC) and caffeine are administered in the same product, added together to a product, added separately to a product, or administered separately. It also covers products that are provided in immediate release form, or in any time-released or sustained methods.

Exemplary Receptor Switcher Dosages

In addition, for certain embodiments of this invention, the preferred Receptor Switcher is N-acetyl-cysteine (NAC). Successful preliminary trials and case studies have been done using about 150-about 1500 mg daily, but lower doses are likely to be effective for certain formulations and conditions. In other embodiments of the invention, the daily dosage of NAC is selected from the group consisting of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or above 1500 mg. Regarding combinations of NAC and caffeine, the parent application suggested that for the treatment of Emotional Distress as well as Addiction, Dependence, and Cravings (e.g., Drugs, Alcohol, Food, Behavior) the administration of NAC (about 400-about 1200 mg)+Caffeine (about 25-about 200 mg) would be appropriate.

### II. Background Regarding Distress Dysfunction

A. Description of Distress Dysfunction

Example 1 below describes a study in which one of the following Endorphin Enhancers was used in relatively low dose—caffeine (about 50-about 200 mg), theophylline (100-300 mg), rolipram (about 1-about 50 μg), and forskolin (200 mg)—in combination with a Receptor Switcher, either an ultra-low-dose of naltrexone (1-100 μg) (ULDN) or a moderately low-dose of magnesium sulfate (0.7-5 g). In addition, each of these agents was administered alone, to determine the potential synergistic effects of co-treatment. Spontaneous, unsolicited reports from the majority of subjects in these trials revealed an unexpected set of therapeutic benefits from these co-treatment formulations, separate and distinct from the issue of nociceptive pain relief which was, at the outset, the sole purpose of the study. Completely unplanned and unexpected was the entirely new discovery that these agents, in all co-treatment formulations, dramatically reduced a surprising variety of symptoms in the subjects separate and distinct from nociceptive pain, including anxiety and depressed mood, gastrointestinal disturbances, emotional and physical agitation, impulsive anger, premature ejaculation, drug cravings, and PMS symptoms. In further studying this phenomenon, another surprising pattern emerged. The formulations were most effective in reducing these non-pain-related symptoms in subjects who initially had relatively low pain thresholds and tolerance prior to taking the drugs, suggesting underlying hyperalgesia.

Moreover, in addition to validating the existence of Distress Dysfunction, the elusive treatment for emotional and physical distress that Dr. Steven Crain had been searching for had suddenly been discovered. This pivotal study, as well as subsequent research and clinical case studies, suggested that a safe and effective treatment for a wide variety of clinical disorders and symptoms had unexpectedly been discovered. [See Examples 1-12.] This treatment could consist of surprisingly low doses of specific agents that, when combined using principles that are based on this novel understanding of the stress response neurotransmitter systems, have a dramatic and synergistic effect on reducing hyperalgesia as well as a variety of other noxious symptoms of Distress Dysfunction. Specifically, the combination of a Receptor Switcher, such as ULDN, VLDN, MSM, NAC, or magnesium sulfate, with an Endorphin Enhancer, such caffeine, simultaneously blocked protracted excitatory opioid signaling and increased endorphin levels, thereby reducing emotional and physical distress and enhancing a sense of well-being.

B. Treatment for Distress Dysfunction

Effective endorphinergic treatment of Distress Dysfunction includes both: (1) at least one Class I agent that restores and maintains stress-related neurotransmitter receptors in a basal inhibitory mode (Receptor Switcher), and (2) at least one of the following (a) one or more Class II agents that restore, maintain, and release sufficient levels of endogenous opioids (i.e., endorphins) for healthy functioning (Endorphin Enhancers), and/or (b) one or more Class III agents that bind with opioid receptors (i.e., exogenous opioid agonists) (Exogenous Opioids), and/or (c) one or more Class IV agents that have a synergistic effect with the endogenous opioid system as well as related neurotransmitter systems (Synergistic Enhancers). Treatment methods that do not restore and maintain balance in both receptor mode and neurotransmitter levels are not only insufficient, but can actually exacerbate imbalances and, therefore, distress symptoms in many individuals. A visual comparison of the dramatically different effects of the balanced cotreatment formulations taught by this invention and conventional unbalanced formulations is shown in the figures.

### III. Definitions

The present invention is described herein using several definitions, as set forth below and throughout the application. The terms defined herein are used in the singular and plural as context indicates.

As used herein, the term “about”, will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The term “active agent” is used herein to refer to a chemical material or compound that induces a desired effect when administered topically or subcutaneously, and includes agents that are therapeutically and/or prophylactically effective as pharmaceuticals (“pharmacologically active agents”), as well as agents that are cosmeceutically effective (“cosmeceutically active agents”). Also included are derivatives and analogs of those compounds or classes of compounds specifically mentioned that also induce the desired effect. An “effective” amount of an active agent is a nontoxic but sufficient amount of an active agent to provide the desired beneficial effect. More specifically, a “therapeutically effective,” “prophylactically effective,” or “cosmeceutically effective” amount is a nontoxic but sufficient amount of a beneficial agent to provide the desired therapeutic, prophylactic, or cosmeceutical effect.

“Distress” means emotional and/or physical pain or suffering affecting the body, behavior, and/or the mind.

“Distress dysfunction” means a constellation of conditions, symptoms, and disorders, whether (a) the result of an endogenous condition or (b) the result or side effect of an exogenous medication, drug, or other agent, wherein a component thereof is the presence of one or more of the following: (a) signs or symptoms of distress (and/or a diminution of happiness, pleasure, contentment and a positive sense of well being), which interferes with an individual's quality of life and functioning, or (b) unpleasant or deleterious side effects of a medication, drug, or other agent, which may, or may not, interfere with its potential therapeutic benefits. Distress Dysfunction includes, but is not limited to, the following conditions, symptoms and/or disorders: (1) Anxiety Disorders, including, but not limited to, Panic Disorders, Agoraphobia, Specific Phobias, Social Phobias, Obsessive-Compulsive Disorder, Post-Traumatic Stress Disorder, Acute Stress Disorder, Generalized Anxiety Disorder, Substance-Induced Anxiety, Anxiety Related to Medical Disorders, Anxiety Disorder Not Otherwise Specified (NOS), as well as signs and symptoms of anxiety, stress, agitation, and worry that are not classified as an Anxiety Disorder; (2) Mood Disorders, including, but not limited to, Depressive Disorders, Dysthymic Disorder, Bipolar I Disorder, Bipolar II Disorder, Bipolar Disorder NOS, Cyclothymic Disorder, Mood Disorders Related to Medical Conditions, Seasonal Affective Disorder, Mood Disorders NOS, as well as signs and symptoms of depressed mood, anhedonia, despair, anhedonia, hypomania, mania, and negative hedonic tone that are not classified as a Mood Disorder; (3) Somatoform Disorders, including, but not limited to, Somatization Disorder, Somatoform Disorder, Conversion Disorder, Pain Disorder Associated with Psychological Factors, Pain Disorder Associated with Medical Conditions, Hypochondriasis, Body Dysmorphic Disorder, and Somatoform Disorder NOS; (4) Factitious Disorders, including but not limited to, Factitious Disorders with Psychological Signs and Symptoms, Factitious Disorders with Physical Signs and Symptoms Factitious Disorders with Combined Psychological and Physical Signs and Symptoms, and Factitious Disorder NOS; (5) Dissociative Disorders; (6) Sexual Dysfunction, including, but not limited to, Sexual Desire Disorders, Sexual Arousal Disorders, Orgasmic Disorders, Premature Ejaculation, Erectile Dysfunction, Sexual Pain Disorder, Sexual Dysfunction to a General Medical Condition, Substance-Induced Sexual Dysfunction, Sexual Dysfunction NOS, as well as signs and symptoms of sexual dissatisfaction and dysfunction that are not classified as a Sexual Dysfunction disorder; (7) Eating Disorders, including, but not limited to, Bulimia Nervosa, Anorexia Nervosa, Binge Eating, Eating Disorder NOS, as well as signs and symptoms of eating and appetite problems that are not classified as an Eating Disorder; (8) Gastrointestinal Disorders, including, but not limited to, Irritable Bowel Syndrome (IBS) with Predominately Diarrhea, IBS with Predominately Constipation, and IBS Mixed Type, Crohn's Disease, as well as GI distress including, but not limited to, nausea, vomiting, diarrhea, constipation, and bloating; (9) Pre-Menstrual Syndrome (PMS) and other hormonally-related distress signs and symptoms; (9) Movement Disorders, including, but not limited to, Restless Leg Syndrome; (10) Fibromyalgia; (11) Sleep Disorders, including, but not limited to, Insomnia, Dyssomnias Parasomnias as well as signs and symptoms of sleep problems that are not classified as a Sleep Disorder; (12) Impulse-Control Disorders, including, but not limited to, Intermittent Explosive Disorder, Kleptomania, Pyromania, Pathological Gambling, Trichotillomania, Impulse Control Disorder NOS as well as signs and symptoms of impulsivity that are not classified as an Impulse-Control Disorder; (13) Psychological Factors Affecting Medical Conditions; (14) Medication-Induced Movement Disorders; (15) Alcohol-Related Disorders, including, but not limited to, Alcohol Dependence, Alcohol Abuse, Alcohol Addiction, Alcohol-Induced Disorders, Alcohol-Related Disorder NOS as well as alcohol-related problems that are not classified as an Alcohol-Related Disorder; (16) Opioid-Related Disorders, including, but not limited to, Opioid Dependence, Opioid Addiction, Opioid Abuse, Opioid-Induced Disorders, Opioid-Related Disorder NOS, as well as opioid-related problems that are not classified as an Opioid-Related Disorder; (17) Caffeine-Related Disorders, including, but not limited to, Caffeine Dependence, Caffeine Addiction, Caffeine Abuse, Caffeine-Induced Disorders, Caffeine-Related Disorders NOS as well as caffeine-related problems that are not classified as a Caffeine-Related Disorder; (18) Cannabis-Related Disorders, including, but not limited to, Cannabis Dependence, Cannabis Addiction, Cannabis Abuse, Cannabis-Induced Disorders, and Cannabis-Related Disorder NOS; (19) Amphetamine (or Amphetamine-Like)-Related Disorders, including but not limited to, Amphetamine Dependence, Amphetamine Addiction, Amphetamine Abuse, Amphetamine-Induced Disorders, and Amphetamine-Related Disorder NOS; (20) Cocaine-Related Disorders, including, but not limited to, Cocaine Dependence, Cocaine Addiction, Cocaine Abuse, Cocaine-Induced Disorders, and Cocaine-Related Disorder NOS; (21) Nicotine-Related Disorders, including, but not limited to, Nicotine Dependence, Nicotine Addiction, Nicotine Abuse, Nicotine-Induced Disorders, and Nicotine-Related Disorder NOS; (22) Inhalant-Related Disorders, including, but not limited to, Inhalant Dependence, Inhalant Addiction, Inhalant Abuse, Inhalant-Induced Disorders, and Inhalant-Related Disorder NOS; (23) Phencyclidine-Related Disorders, including, but not limited to, Phencyclidine Dependence, Phencyclidine Addiction, Phencyclidine Abuse, Phencyclidine-Induced Disorders, and Phencyclidine-Related Disorder NOS; (24) Sedative-, Hypnotic-, or Anxiolytic-Related Disorders, including, but not limited to, Sedative-, Hypnotic-, or Anxiolytic Dependence, Addiction, and/or Abuse, Sedative-, Hypnotic-, or Anxiolytic-Induced Disorders, and Sedative-, Hypnotic-, or Anxiolytic-Related Disorder NOS; (25) Polysubstance-Related Disorders; (26) Pervasive Developmental Disorders, including, but not limited to, Autism Disorder, Rhett's Disorder, Asperger's Disorder, or Pervasive Developmental Disorder NOS; (27) Attention-Deficit and Disruptive Behavior Disorders, including, but not limited to Attention-Deficit/Hyperactivity Disorder, Conduct Disorder, Oppositional Disorder, Disruptive Behavior Disorder NOS as well as attentional and concentration problems that are not classified as an Attention-Deficit Disorder; (28) Chronic Fatigue Disorder; (29) Behavioral addictions, compulsions, and dysfunctions, including, but not limited to, sex, pornography, gambling, shopping, eating, drinking, smoking, computer use, and cleaning; (30) Pain disorders, including, but not limited to, distressing, neuropathic, nociceptive, migraine and psychogenic pain; (31) Psychotic disorders, including, but not limited to, schizophrenia; (32) Unpleasant or deleterious side effects of CLASS II, III, or IV agents when such agents are administered alone (i.e., without co-administration with an CLASS I agent) which may, or may not, interfere with the potential therapeutic benefits of CLASS II, III, or IV agents, including, but not limited to PDE inhibitors, opioid and non-opioid analgesics, stimulants, SSRIs, SNRIs, NGF, and amino acids; (33) Respiratory disorders including, but not limited to, asthma, COPD, neonatal apnea; (34) Nerve damage and neuropathy including, but not limited to, HIV-induced and diabetic neuropathy; (35) Inflammatory disorders; (36) Allergic and non-allergic glutamate and mono-sodium glutamate related disorders, including “Chinese Food Syndrome”; and (37) Emotional and physical malaise, distress, discomfort, pain, restlessness, irritability, worries, cravings, compulsions, obsessions, agitation, addictions, and other related complaints and signs of protracted negative hedonic tone that may, or may not, be part of a traditional medical or psychiatric disorder. Distress Dysfunction is not limited to these conditions and diagnoses and is best defined by a variety of symptoms, conditions, syndromes, and disorders, characterized by dysfunctional emotional and physical distress and pain.

“Distressing pain” means an exaggerated emotional and/or physical reaction to perceived, anticipated and/or real danger or harm, which typically includes hyperalgesia and hypersensitivity to perceived or real injury.

“Endorphin enhancers” (CLASS II AGENTS) means an exogenous agent that directly or indirectly enhances the production, release and/or functioning of endogenous opioids, i.e., endorphins, and/or inhibits their reuptake. CLASS II agents are functionally defined such that, when co-administered with CLASS I agents, they reduce and/or resolve Distress Dysfunction symptoms. CLASS I agents include, but are not limited to, cyclic adenosine monophosphate (cAMP) phosphodiesterase (PDE) inhibitors or agents that directly enhance cAMP, a cAMP phosphodiesterase (PDE) inhibitor, an agent that directly enhances cAMP, a specific or non-specific cAMP PDE inhibitor, a specific cAMP PDE-4 inhibitor, ginkgo biloba, theophylline, roflumilast, ibudilast, cilomilast, ardenafil, tadalafil, sildenafil, zaprinast, rolipram, methylxanthine, milrinone, inaminone, cilostazol, caffeine, guarana, ginkgo biloba, forskolin, celecoxib, excitatory amino acids, a salt of an excitatory amino acid, all forms of excitatory amino acids, glutamic acid, aspartic acid, glutamine, mono-sodium glutamate (MSG), N-methyl-D-asparate (NMDA), phenylalanine, dl-phenylalanine (DLPA), and nerve growth factor (NGF).

“Exogenous opiods” (CLASS III AGENTS) means exogenous agents that activate and/or bind with opioid receptors, triggering inhibitory and/or excitatory signaling. CLASS III agents include, but are not limited to, exogenous opioid agonists (full, partial, mixed), tramadol, morphine, oxycodone, hydrocodone, papaverine, codeine, dihydrocodeine, fentanyl, hydromorphone, buprenorphine, butorphanol, methadone, loperamide, alfentanil, levorphanol, menthol, meperidine, nalbuphine, oxymorphone, pentazocine, pentazocine, propoxyphene, remifentanil, and sufenta.

“Hedonic tone homeostasis” means the tendency of the opioidergic, serotonergic, dopaminergic and related neurotransmitter systems to maintain positive hedonic tone, when adaptive, and to restore positive hedonic tone following stressful conditions and stimuli, which may acutely produce negative hedonic tone.

“Homeostasis” means the tendency of an organism to actively regulate its internal conditions, usually by a system of feedback controls, so as to stabilize health and functioning, regardless of the changing conditions, as well as the ability of the body to actively seek and maintain a condition of equilibrium or stability within its internal environment when dealing with external changes.

“Low-dose naltrexone” means a dose of about 500 μg to about 1000 μg.

“Negative hedonic tone” means the diminution of happiness, pleasure, and contentment, and is typically associated with the experience of emotional and/or physical distress and alert.

“Positive hedonic tone” means a positive sense of well-being, happiness, pleasure and contentment.

“Receptor switchers” (CLASS I AGENTS) means an exogenous agent that blocks opioid excitatory receptor signaling, thereby switching protracted excitatory mode to homeostatic basal inhibitory mode. CLASS I agents are functionally defined such that, when co-administered with CLASS II, III, and/or IV agents, reduce and/or resolve Distress Dysfunction symptoms. CLASS I agents include, but are not limited to: ultra-low-dose, very-low-dose and low-dose opioid antagonists, ultra-low-dose, very-low-dose, and low-dose naltrexone, naloxone, diprenorphine, nalmefene, and norbinaltorphimine, agents that inhibit synthesis or activity of GM1 ganglioside, neuraminidase inhibitors, agents that increase sulfates in the body, methylsulfonylmethane (MSM), magnesium sulfate, chondroitin sulfate, n-acetyl-cysteine (NAC), oseltamivir, zanamivir, laninamivir, peramivir, scutellaria, and 5,7,4′-trihydroxy-8-methoxyflavone.

“Synergistic enhancers” (CLASS IV AGENTS) means exogenous agents that have a synergistic effect with the endogenous opioid system. CLASS IV agents are functionally defined such that, when co-administered with CLASS I agents, they reduce and/or resolve Distress Dysfunction symptoms. The addition of CLASS II and/or III agents may further enhance the therapeutic effects of a combination of CLASS I and IV agents. CLASS IV agents include, but are not limited to, agents that support the functioning, production and release of endogenous opioid, serotonin, dopamine, epinephrine, norepinephrine, and glutamate neurotransmitters, non-opioid analgesics, non-steroidal anti-inflammatory drugs (NSAIDs), acetaminophen, celecoxib, white willow bark, acetylsalicylic acid, salicin, ibuprofen, naproxen, ketoprofen, indomethacin, fenoprofen, tolmetin, sulindac, meclofenamate, piroxicam, flurbiprofen, diclofenac, stimulants, selective serotonin reuptake inhibitors (SSRI), serotonin agonists, antagonists and modulators, selective norepinephrine reuptake inhibitors (SNRIs), citalopram, dapoxetine, escitalopram, fluoxetine fluvoxamine, paroxetine, sertraline, fluvoxamine, zimelidine, dapoxetine, alosetron, ondansetron, granisetron, bemesetron, eplivanserine, deramciclane, agomelatine, elazasonan, pruvanserin, asenapine, zomari, valazodone, bifeprunox, buspirone, ritanseron, geperone, paliperidone, clomipram, doxepin, haloperidol, risperidone, methylphenidate, amino acids, a salt of an inhibitory amino acid, all forms of amino acids, gamma-aminobutrynic acid (GABA), PharmaGABA, glycine, taurine, tryptophan, 5HTP, phenylalanine, dl-phenylalanine (DLPA), acetyl-L-carinitine (ALC), valine, threonine, methionine, lysine, leucine, isoleucine, tyrosine, alanine, arginine, histidine, serine, selenocfysteine, proline, glycine, cysteine, aspargine, alanine, S-adenosylmethionine (SAMe), cannabis, all forms and derivatives of cannabis, L-DOPA, vitamins and minerals, luteolin, quercetin, qercetin-3-O-methylether (3-MQ, 2), quercetin-3,7,4′-O-trimethylether, ayanin, quercetin-3,7,3′,4′-O-tetramethylether, quercetin-3,5,7,3′,4′-O-petamethylether, quercetin-3,5,7,3′,4′-O-pentaacetate, quercetin-3-O-methyl-5,7,3′,4′-O-tetraacetate, methylcobalamin, vitamin C, vitamin D, vitamin D-3-vitamins B1, B2, B3, B6, and B12, folic acid, niacin, or niacinamide, folinic acid, calcium folinate, methylcobalamin, pyridoxal-5′-phosphate (P5P), alkaloids, flavonoids, and saponins, hesperetin, hesperidin, naringin, naringenin, epigallocatechin-3-gallate (EGCG), dioclein, genistein, daidzein, eriodictyol, prunetin, biochanin A, apigenin, myricetin, liquiritigenin, liquiritin, kaempferol, isoliquiritigenin, chrysin, rutin, cyanidin, delphinidin, pelargonidin, isorhamnetin, vitamin C, St. John's Wort, passion flower, hyperforin, hypericin, biotin, vitamin B5 (pantothenic acid), magnesium, alpha-ketoglutarate, copper, zinc, L-theanine, iron, california poppy, ginseng (Panax spp.), licorice, night-blooming cereus (Selenicereus grandiflorus; Cactus grandiflorus), hordenine, nutmeg, myristicin, tyramine, scotch broom, green tea, ephedra, yohimbe, myrrh, boswellia, frankincense, peppermint oil, and menthol.

“Very-low-dose naltrexone” means a dose of about 125 μg to about 500 μg.

“Ultra-low-dose naltrexone” means a dose of about 1 μg to about 125 μg.

### IV. Components of the Compositions of the Invention

The compositions of the invention comprise components which act synergistically to treat, prevent, mitigate, and/or reduce the side effects of caffeine consumption. Specifically, the compositions of the invention, as defined herein, comprise at least one Receptor Switcher in combination with caffeine.

This invention also teaches that formulations that contain Receptor Balancers (e.g., ultra-low-dose and very-low-dose opioid antagonists and/or neuraminidase inhibitors) alone, or in combination with Endorphin Enhancers (e.g., PDE inhibitors or excitatory amino acids) and/or Synergistic Enhancers (e.g., amino acids), can be administered therapeutically when an individual is already taking certain Synergistic Enhancers (e.g., stimulants, SSRI or SNRI) or Exogenous Opioids (e.g., tramadol, oxycodone or morphine) to enhance their therapeutic benefit, reduce side effects, and minimize the dose of the medications.

A. Class 1 Agents: Receptor Switchers

Regarding agents that switch stress-related neurotransmitter receptors from a protracted excitatory mode to a basal inhibitory mode, two categories of agents have been discovered that function for this purpose: (1) ultra-low doses, very-low doses, and low-doses of opioid antagonists; and (2) agents that inhibit synthesis or activity of GM1-ganglioside. Opioid antagonists include, but are not limited to, naltrexone, naloxone, norbinaltorphimine, diprenorphine, and similarly acting opioid peptides and alkaloids. Agents that inhibit synthesis or activity of GM-1 ganglioside include, but are not limited to, neuraminidase inhibitors, agents that increase sulfates in the body, magnesium sulfate, sodium sulfate, chondroitin sulfate, n-acetyl cysteine (NAC), methylsulfonylmethane (MSM), oseltamivir, zanamivir, laninamivir, peramivir, scutellaria, 5,7,4′-trihydroxy-8-methoxyflavone, and similarly acting neuraminidase inhibitors.

While prior teachings suggest that these classes of agents block or inhibit excitatory signaling in the endogenous opioid system, the discoveries underlying this invention suggest that these agents have synergistic serotoninergic, dopaminergic, epinephrinergic, norepinephrinergic, and glutamatergic effects, given the homeostatic interconnectedness of the stress-related neurotransmitter systems. In addition, while not characterized in prior arts as a neuraminidase inhibitor, the anti-viral qualities of n-acetyl-cysteine (NAC), in addition to the case studies leading to this invention, suggest that NAC functions remarkably well as a Receptor Switcher. In addition, agents that increase the levels of sulfates in the body have been determined by our trials and case studies to function well as Receptor Switchers, including, but not limited to, methylsulfonylmethane (MSM). Magnesium sulfate can be very useful as a Receptor Switcher. While the sulfate is the critical component that switches receptor mode, magnesium sulfate provides the additional benefits of magnesium, which can further reduce distress as well as certain GI problems, such as constipation.

Therefore, these two categories of agents, ultra-low-dose, very-low-dose, and low-dose opioid antagonists and GM1 ganglioside inhibitors, are used in this invention for the functional Class I agents that selectively switch stress-related neurotransmitter receptors from a protracted excitatory mode to a basal inhibitory mode. This invention includes these and any other agents that inhibit or block excitatory receptor signaling and/or enhance inhibitory receptor signaling in the opioid, serotonin, dopamine, glutamate, epinephrine, and/or norepinephrine neurotransmitter systems. Alone, these Class I agents have less impact on reducing dysfunctional distress and nocieptive pain compared to the synergistic benefits that result from combining Class I agents with Class II and/or III and/or IV agents. In fact, in many cases, Receptor Switchers, when administered alone, have minimal or no therapeutic impact on reducing symptoms of Distress Dysfunction.

Preferred Receptor Switchers are ultra-low-dose and very-low-dose naltrexone and naloxone as well as n-acetyl-cysteine (NAC), magnesium sulfate, and methylsulfonylmethane (MSM). All of these agents have been shown to have very surprising and dramatic synergistic effects in the cotreatment formulations studied using the principles taught by this invention. They also are all known for being remarkably safe and provide additional therapeutic benefits. Since they all appear to be effective in combination with a variety of agents, which are known to enhance different neurotransmitter systems, and these combination have been shown to reduce a variety of symptoms, including both physical and emotional dysfunctional distress, these Receptor Switchers appear to function in a similar way. This set of findings also suggests that they restore basal opioid receptor inhibitory signaling in such a way that the Receptor Switcher impacts a variety of stress-related neurotransmitter systems. The choice of Receptor Switcher can be made dependent on a variety of factors including further studies to determine maximum benefit for different indications NAC, MSM, and magnesium are preferred agents for non-prescription and nutraceutical formulations for the treatment and prevention of Distress Dysfunction.

For certain embodiments of this invention, the preferred Receptor Switchers are naltrexone and naloxone.

For certain embodiments of this invention, the preferred Receptor Switcher is ultra-low-dose naltrexone (about 125 micrograms or less daily). Our trials and case studies have demonstrated remarkable effectiveness using about 1-about 125 micrograms of ULDN. Evidence suggests that while doses of about 5 micrograms can be remarkably effective for certain indications and populations, the higher end of this dosing range (about 100-about 125 micrograms) appears to be more reliable with more consistent therapeutic benefits over time for more people. In other embodiments of the invention, the daily dosage of naltrexone can be selected from the group consisting of about 0.001, about 0.01, or about 0.1 micrograms. In other embodiments of the invention, the daily dosage of naltrexone can be selected from the group consisting of about 1, about 2, about 3, about 4, about 5, about 10, about 15, about 20, about 25, about 65, about 70, about 75, about 80, about 85, about 90, about 95, about 100, about 105, about 110, about 115, about 120, or about 125 micrograms.

For certain embodiments of this invention, the preferred Receptor Switcher is very-low-dose naltrexone (about 125-about 500 micrograms daily). Validation studies for this invention suggest that the low end of VLDN dosing (about 125-about 150 micrograms) results in maximal and consistent therapeutic benefits for certain conditions. In other embodiments of the invention, the daily dosage of naltrexone can be selected from the group consisting of about 125 about 130, about 135, about 140, about 145, about 150, about 155, about 160, about 165, about 170, about 175, about 180, about 185, about 190, about 195, about 200, about 205, about 210, about 215, about 220, about 225, about 250, about 275, about 300, about 350, about 400, about 450, or about 500 micrograms.

For certain embodiments of this invention, the preferred Receptor Switcher is low-dose naltrexone (about 500-about 1000 micrograms daily). In other embodiments of the invention, the daily dosage of naltrexone can be selected from the group consisting, about 550, about 600, about 650, about 700, about 750, about 800, about 850, about 900, about 950, or about 1000 micrograms.

For certain embodiments of this invention, the preferred Receptor Switcher is n-acetyl-cysteine (NAC). Successful preliminary trials and case studies have been done using about 300-about 1200 mg daily, but lower doses are likely to be effective for certain formulations and conditions. In other embodiments of the invention, the daily dosage of NAC is selected from the group consisting of about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or above 1500 mg.

For certain embodiments of this invention, the preferred Receptor Switcher is magnesium sulfate. Successful preliminary trials and case studies have been done using about 25-about 1000 mg daily, but lower and higher doses are likely to be effective for certain formulations and conditions. In other embodiments of the invention, the daily dosage of magnesium sulfate is selected from the group consisting of about 5, about 10, about 15, about 20, about 25, about 50, about 100, about 150, about 200, about 250, about 300, about 350, about 400, about 450, about 500, about 550, about 600, about 650, about 700, about 750, about 800, about 850, about 900, about 950, or above 1000 mg.

For certain embodiments of this invention, the preferred Receptor Switcher is methylsulfonylmethane (MSM). Successful preliminary trials and case studies have been done using about 100-about 1000 mg daily, but lower and higher doses are likely to be effective for certain formulations and conditions. In other embodiments of the invention, the daily dosage of MSM is selected from the group consisting of about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or above 1500 mg.

Validation studies of this invention have suggested that ultra-low-dose and very-low-dose naltrexone is particularly effective in most oral cotreatment formulations for a wide variety of Distress Dysfunction symptoms. Given oral bioavailabiity of naltrexone, evidence suggests that while remarkably low doses of naltrexone, of 5 micrograms or less can be effective, relatively higher doses of ULDN and VLDN, in the range of 100-150 micrograms appear to be more consistently and reliably therapeutic for more people and conditions over time. Ultra-low-dose, very-low-dose, and low-dose naloxone is the preferred Receptor Balancer for intravenous and sublingual administrations. Lower doses of naloxone are particularly effective given the relative bioavailability of intravenous and sublingual administration. Since the formulations used in this invention involve naltrexone doses that are about 500-1,000 times lower than those used for other indications, ULDN and VLDN is a remarkably innocuous and safe agent for this application. At the same time, since opioid antagonists are effective as Receptor Switchers only at very low doses, and can create Distress Dysfunction at higher doses, it has been difficult to determine the most effective dose ranges. This concern may become particularly problematic in long-term administration since there is evidence to suggest that these agents may accumulate in the body. Therefore, other Receptor Switchers may be preferred for certain applications. Other agents, such as NAC, MSM, and magnesium sulfate, have the advantage of being less dose-dependent. Moreover, since they were in general use in the United States prior to Oct. 15, 1994, they not considered to be “grandfathered dietary supplements” and are not classified as either “drugs” or “new dietary supplements”. ((21 CFR §312.2; 21 USC §350b; 21 USC §355.) Accordingly, they may be offered for sale to the public without the time and expense that would be required to do so with regard to drugs or new dietary supplements. However, NAC is an excellent Receptor Switcher since it has the added benefit of anti-inflammatory, anti-oxidant, and anti-viral effects, which may be important in many applications, as in the treatment of respiratory conditions. MSM is also a preferred Receptor Switcher since is known to have pain-relieving effects as well as an ability to increase concentration and attention. Magnesium sulfate, or Epsom salts, can be useful in certain indications to prevent constipation, such as in the treatment of certain forms of IBS, as well as additional benefits as a calming agent. However, its use should be carefully evaluated since it does have laxative effect.

B. Class II Agents: Endorphin Enhancers

One class of agents that have remarkable synergy with Receptor Switchers are those that enhance the production, release, or functioning of endogenous opioids (Endorphin Enhancers). Cyclic AMP enhancers, particularly specific cAMP PDE-4 inhibitors, such as roflumilast and ginkgo biloba, as well as non-specific cAMP PDE inhibitors, such as theophylline, caffeine, guarana, and ibudilast, enhance the release of cAMP, which, in turn, enhances the release of endogenous opioids (i.e., endorphins). In addition to cAMP PDE inhibitors, less potent cAMP enhancers include excitatory amino acids, such as glutamic acid, as well as forskolin and nerve growth factor (NGF). In addition, endogenous opioid reuptake inhibitors, such as DLPA, which both enhance the release of endogenous opioids as well as block the enzymes that reuptake them, provide an enhanced level of endorphins for longer periods of time. There is also evidence to suggest that celecoxib functions, in part, by enhancing the release of endorphins, making it an important Endorphin Enhancer for certain indications, such as arthritic pain.

When administered alone, Endorphin Enhancers have the potential to produce increased inhibitory signaling. However, since they would typically be administered when injuries or stress are present, or when the receptors are set in an excitatory mode, they are more likely to produce excitatory signaling, thereby iatrogenically triggering symptoms of Distress Dysfunction, such as pain, hyperalgesia, anxiety, and gastrointestinal symptoms, rather than reducing them. Unfortunately, most clinical use of these agents is conducted in the treatment of conditions and diagnoses that are, in fact, forms of Distress Dysfunction but heretofore have not been so identified. This mechanism explains many of the typical side effects seen with these agents. However, when co-administered with Receptor Switchers, Endorphin Enhancers trigger inhibitory signaling, leading to enhanced and prolonged analgesia and well being. Therefore, combining one or more Receptor Switchers, such as ULDN, MSM, NAC, or magnesium sulfate, with one or more Endorphin Enhancers, such as roflumilast, ginkgo biloba, guarana, celecoxib, glutamic acid, caffeine, theophylline, ibudilast, forskolin or NGF, creates a remarkable new generation of non-opioid pharmaceutical and nutraceutical formulations for the treatment of a wide variety of Distress Dysfunctions, by rebalancing the endogenous opioid system, restoring a basal homeostatic inhibitory mode together with normal levels of endorphins.

Regarding agents that enhance the production, release and/or functioning of endogenous opioids (i.e., endorphins), several categories of agents have been discovered that function for this purpose. All of these agents have variable impact on Distress Dysfunction when taken without co-administration with an agent that reduces excitatory opioid receptor signaling (Receptor Switchers). In fact, Endorphin Enhancers can have a negative impact if taken alone when the opioid receptors are in a protracted excitatory mode, which is typical when treating Distress Dysfunction. In this condition, the increased release of endorphins triggered by these agents can result in excitatory signaling, producing increased pain and negative hedonic tone, creating and exacerbating Distress Dysfunction. Without the understanding that is taught by this invention, there was no clear explanation for the highly variable findings of studies and treatments using Endorphin Enhancers alone.

However, when combined with an agent that reduces protracted excitatory signaling (Receptor Switcher), these Endorphin Enhancers have a surprising and dramatic effect on reducing acute and chronic emotional and physical distress. These agents, by enhancing the release of endorphins, facilitate endogenous opioid receptor inhibitory signaling, as long as a Receptor Switcher is present to maintain opioid receptors in the inhibitory mode. This cotreatment formulation is remarkably effective in reducing and/or resolving a wide variety of Distress Dysfunction symptoms, providing positive hedonic tone, including calm, well being, and relief from pain.

Endorphin Enhancers include agents that enhance the release of endogenous opioids (i.e., endorphins) directly, such as forskolin. Preferred Endorphin Enhancers also include agents that enhance endorphins indirectly. Many of these agents trigger the release of cyclic-AMP (cAMP), which in turn enhances the release of endorphins. Endorphin Enhancers that can be effective for this purpose include excitatory amino acids, such as glutamic acid and aspartic acid as well as salts of excitatory amino acids, such as monosodium glutamate (MSG). Glutamic acid and MSG have been shown to be remarkably effective for a wide variety of Distress Dysfunction symptoms when combined with Receptor Switchers in all validation studies for this invention. Certain amino acids, such as phenylalanine and dl-phenylalanine (DLPA), not only trigger the release of endorphins, but also block the enzymes that remove endorphins from the system. DLPA is a particularly effective selective endorphin reuptake inhibitor (SERI) and has been shown to enhance and prolong the therapeutic benefits of a variety of balanced cotreatment formulations in the validation studies for this invention. These amino acids have been widely used for other applications and have an excellent safety profile.

A particularly effective group of agents that trigger the release of cAMP, and therefore endorphins, are cAMP phosphodiesterase (PDE) inhibitors. The most potent of this group are specific cAMP PDE-4 inhibitors, known for their impact on the release of cAMP and, therefore, can be used at remarkably low doses. Therefore, preferred Endorphin Enhancers include specific cAMP PDE-4 inhibitors, such as roflumilast and ginkgo biloba. Roflumilast is a particularly compelling choice since it has been recommended for approval as safe and effective in Europe for the treatment of COPD, and is currently being reviewed in the U.S. for this purpose. Remarkably low doses of roflumilast have been shown to be extremely effective for a wide variety of Distress Dysfunction symptoms, when combined with a Receptor Switcher, in validation studies for this invention. Gingko biloba also functions as a cAMP PDE-4 inhibitor, and has certain benefits as a natural non-prescription agent and is known to improve circulation, memory, and concentration. Gingko biloba has also been shown in our preliminary trials to be quite effective, when combined with Receptor Balancers, such as NAC, for a variety of Distress Dysfunction symptoms, particularly emotional distress, which makes this a preferred non-prescription formulation. Non-specific cAMP PDE inhibitors, such as theophylline, ibudulast, methyxanthines, guarana, caffeine, and isobutylmethylxanthine (IBMX), also function as Endorphin Enhancers, though require higher doses than specific PDE-4 inhibitors. Theophylline and caffeine have been shown to be effective for a variety of Distress Dysfunction symptoms, when combined with Receptor Switchers, in validation studies for this invention. Theophylline has the advantage of decades of clinical use as a prescription medication for asthma. However, guarana is a preferred non-prescription Endorphin Enhancer since it has a more gradual release and longer-acting effects than other agents, such as caffeine, and has other known benefits regarding concentration, attention, and energy. Therefore, for OTC and nutraceutical formulations, preferred Endoprhin Enhancers include guarana, gingko biloba, and DLPA.

For certain embodiments of this invention, the preferred Endorphin Enhancer is caffeine. Successful preliminary trials and case studies have been done using about 25-about 100 mg daily, though higher or lower doses are likely to be effective for certain formulations and indications. In other embodiments of the invention, the daily dosage of caffeine is selected from the group consisting of about 1, about 5, about 10, about 20, about 30, about 40, about 50, about 60, about 70, about 80, about 90, about 100, about 110, about 120, about 130, about 140, about 150, about 160, about 170, about 180, about 190, or about 200 mg.

In certain circumstances, this invention teaches the method that at least one Receptor Switcher can be administered to a subject who is already taking an Endorphin Enhancer. For instance, when cAMP PDE inhibitors, such as theophylline, caffeine, or roflumilast, are administered in the treatment of respiratory conditions, such as COPD, asthma, or neonatal apnea, they can cause various side effects, including emotional and physical distress, hyperalgesia, agitation, anxiety, and GI symptoms, essentially producing Distress Dysfunction. Similar side effects are produced by other Endorphin Enhancers, such as nerve growth factor, celecoxib, caffeine, guarana, ginkgo biloba, MSG, and glutamic acid. The administration of at least one Receptor Switcher, such as ULDN (naltrexone or naloxone), MSM, magnesium sulfate or NAC, can not only reduce and/or eliminate these side effects, but can also reverse these effects, producing greater pain relief and calm.

C. Class III Agents: Exogenous Opioids

A second class of agents that has remarkable synergy with Receptor Switchers is exogenous opioid agonists (Exogenous Opioids). Exogenous Opioid agonist drugs (full, partial, mixed), such as tramadol, oxycodone, and morphine, clearly have a dramatic impact on the endogenous opioid system. Exogenous Opioids act like endogenous opioids, binding with opioid receptors, and their impact depends on the mode of the bimodally-acting opioid receptors. In a balanced system, their impact initially leads to inhibitory signaling, resulting in analgesia and even a sense of well being. However, fairly quickly, this increased inhibitory signaling results in a homeostatic balancing response that includes, through cAMP, a reduction in endogenous opioid levels as well as a receptor shift to the excitatory mode. Over time, this leads to a protracted excitatory receptor mode and diminished endogenous opioid levels, producing chronic pain, hyperalgesia, tolerance, dependence, and addiction as well as emotional and physical distress. These iatrogenic problems are greatly exacerbated when the endogenous opioid system is already in a protracted excitatory mode, resulting more immediately in excitatory signaling, leading to an exacerbation of pain and hyperalgesia, tolerance, as well as negative hedonic mode. Thus, while often initially therapeutic, Exogenous Opioids can rapidly lead to the development of serious and significant Distress Dysfunction, even long after the Exogenous Opioids are discontinued.

However, by administering one or more Receptor Switchers with one or more Exogenous Opioids, excitatory signaling is minimized, resulting in enhanced analgesia as well as a dramatic reduction in protracted excitatory mode conditions, reducing and/or eliminating many of the noxious effects of Exogenous Opioids, including hyperalgesia, tolerance, dependence, addiction, and other side effects. Therefore, if a particular patient is determined to require administration of an Exogenous Opioids, these should only be co-administered with Receptor Switchers in the treatment of Distress Dysfunction conditions, including distressing pain, addiction, anxiety, and depression. Given the inherent problems with Exogenous Opioids, however, formulations that primarily include Receptor Switchers with Endorphin Enhancers are preferable, thereby utilizing endogenous opioids rather than exogenous opioids that can both interfere with the body's natural opioid system and, as a result, cause significant, undesirable side effects. However, low-dose Exogenous Opioids can be added to these formulations, with remarkable synergistic effects, for particularly severe and resistant symptoms. Therefore, this discovery leads to the development of a new generation of remarkably safe and effective pharmaceutical formulations for the treatment of Distress Dysfunctions, including severe chronic distressing pain and addiction, that include the combination of Receptor Switchers, such as ULDN, VLDN, NAC, MSM and/or magnesium sulfate, Endorphin Enhancers, such as roflumilast, ginkgo biloba, guarana, DLAP, and/or caffeine, and low-dose Exogenous Opioids, such as tramadol, morphine, hydrocodone, codeine, and/or oxycodone. Compared to Exogenous Opioids administered alone, these formulations are also equally or more potent analgesics for the treatment of nociceptive pain while, at the same time, they reduce noxious opioid side effects

Exogenous opioid agonists bind with opioid receptors in ways similar to endogenous opioids, thereby triggering excitatory or inhibitory signaling, depending on the mode of the receptor. However, the use of these Exogenous Opioids can create serious problems since they tend to inhibit the release of endogenous opioids and lock the opioid receptors in an excitatory mode over time. Given the well-known side effects of exogenous opioid agonists such as oxycodone and morphine, preferred formulations for the treatment of Distress Dysfunction consist of at least one Receptor Switcher with at least one Endorphin Enhancer. This method uses the natural healing power of balancing opioid receptors and neurotransmitters, maximizing use of endorphins in resolving Distress Dysfunction. This endorphinergic treatment has been shown to be remarkably effective in reducing and/or resolving a wide variety of Distress Dysfunction symptoms, including moderate-to-severe distressing pain.

However, there may be circumstances in which the endogenous opioids may be insufficient to effectively resolve symptoms. In this case, low-dose exogenous opioid agonists (Exogenous Opioids) can be added to the combination of Receptor Switcher and Endorphin Enhancer, to supplement the endogenous opioids (i.e., endorphins). The evidence strongly suggests that, as long as at least one Receptor Switcher is included in the formulation, most noxious side effects of the Exogenous Opioid are reduced and/or eliminated including tolerance, dependence, hyperalgesia, and GI symptoms. However, the preferred treatment includes at least one Receptor Switcher and Endorphin Enhancer with an Exogenous Opioid. There is also evidence to suggest that this formulation enhances the analgesic potency of the Exogenous Opioid for nociceptive pain.

Exogenous Opioids include tramadol, morphine, oxycodone, hydrocodone, papaverine, codeine, dihydrocodeine, fentanyl, hydromorphone, buprenorphine, butorphanol, methadone, alfentanil, loperamide, levorphanol, meperidine, nalbuphine, oxymorphone, pentazocine, pentazocine, propoxyphene, remifentanil, and sufenta. While a much weaker opioid, tramadol is a preferred Exogenous Opioid since it has therapeutic benefits through both the endogenous opioid and serontergic neurotransmitter systems, through the Gᵢ coupling. Evidence suggests that, in addition to mild analgesia, tramadol has anti-depressant characteristics. Therefore, tramadol, has great potential in formulations for the treatment of Distress Dysfunction, since both the analgesic and mood enhancing effects are likely to be potentiated by cotreatment with Receptor Switchers through G₁. These synergistic therapeutic benefits of cotreatment with tramadol have consistently been demonstrated in our validation studies. Tramadol has the additional benefit have having a much better side effect profile than most opioids and, as result, does not have the regulatory restrictions of most other opioids, such as oxycodone and morphine. At the same time, low-dose oxycodone is still a preferred Exogenous Opioid since the balanced cotreatment formulation has been shown to enhance its pain relieving effects, while dramatically reducing its side effects. Low-dose morphine is also preferred in balanced cotreatment formulations, using ultra-low-dose naloxone, for intravenous and sublingual administration.

D. Class IV Agents: Synergistic Enhancers

A third agent class that has remarkable synergy with Receptor Switchers includes a variety of agents having a synergistic effect with the endogenous opioid system through the Gᵢ-mediated metabolic processes that trigger the inhibition of pain-sensory neurons (Synergistic Enhancers). There is evidence to suggest that higher levels of Gi, which are produced by enhanced inhibitory signaling potentiate the pain-relieving effects of non-opioid analgesics, such as NSAIDs and acetaminophen. Therefore, there is a synergistic potentiation produced by the combination of non-opioid analgesics, such as acetaminophen, celecoxib, ibuprofen, aspirin, or white willow bark, with Receptor Switchers, such as ULDN, VLDN, MSM, NAC, or magnesium sulfate, creating a new generation of enhanced non-opioid analgesics.

These novel formulations can be further potentiated by combining one or more Receptor Switchers, such as ULDN, VLDN, LDN, MSM, magnesium sulfate or NAC, together with an Endorphin Enhancer, such as roflumilast, caffeine, guarana, DLPA, glutamic acid or ginkgo biloba, and a Synergistic Enhancer, such as acetaminophen, ibuprofen, aspirin, celecoxib or white willow bark.

Similarly, synergistic potentiation occurs with selective serotonin reuptake inhibitors (SSRIs) and selective norepinephrine reuptake inhibitors (SNRIs), such that increased relief from depression, anxiety, distressing pain, addiction, and other Distress Dysfunctions is produced by the combination of SSRIs and SNRIs, such as escitalopram oxalate and venlafaxine, with Receptor Switchers, such as ULDN, MSM, magnesium sulfate, or NAC. Therefore, a new generation of enhanced SSRI/SNRI formulations for depression, anxiety, pain, and addiction are created by this invention. In addition to SSRIs and SNRIs, inhibitory serontonergic and adrenergic agents can function as Synergistic Enhancers.

E. Exemplary Commercial Formulations and Application

There is evidence to suggest that ULDN and VLDN have a synergistic effect through the Gᵢ metabolic process in addition to its function as a Receptor Switcher, making ULDN and VLDN particularly powerful agents in all cotreatment formulations. Similarly, certain multi-function agents, such as tramadol, have particularly potent therapeutic effects in these novel pharmaceutical formulations since they function as both Exogenous Opioids and Synergistic Enhancers. Tramadol has the added advantage of not being listed by the FDA as a controlled substance. Therefore, given their relative potency in cotreatment formulations, ULDN, VLDN, tramadol, and roflumilast, are preferred agents in formulations for certain indications. Celecoxib has the added advantage of being both an Endorpin and a Synergistic Enhancer. At the same time, over-the-counter agents, such as NAC, MSM, magnesium sulfate, guarana, gingko biloba, caffeine, white willow bark, ibuprofen, acetaminophen, SAMe, and specific amino acids, such as glutamic acid, GABA, and 5HTP, are useful in non-prescription formulations.

The following is a list of exemplary indications and dosing with regard to potential commercial applications of this invention. While certain daily dosage examples are suggested below for purposes of illustration (oral administration, unless otherwise noted), potential dose ranges as well as modes of administration are described in more detail elsewhere in this patent.

1. Emotional Distress

(e.g., Excessive Worries, Fears, Irritability, Anger, Agitation)

ULDN (about 1-about 125 μg)+Caffeine (about 25-about 200 mg);
VLDN (about 125-about 500 μg)+Caffeine (about 25-about 200 mg);
NAC (about 400-about 1200 mg)+Caffeine (about 25-about 200 mg);
MSM (about 100-about 800 mg)+Caffeine (about 25-about 100 mg);
Magnesium Sulfate (about 100-about 800 mg)+Caffeine (about 25-about 100 mg).

2. Addiction, Dependence, and Cravings

(e.g., Drugs. Alcohol, Food, Behavior)

### [Formulations for Emotional Distress and Mood and Anxiety Disorders in Addition to:]

ULDN (about 1-about 125 μg+Caffeine (about 25-about 200 mg);
VLDN (about 125-about 500 μg)+Caffeine (about 25-about 200 mg);

3. Physical Distress

(e.g., Distressing Pain, Hypersensitivity, and Hyperalgesia)

[Formulations for Emotional Distress and Mood and Anxiety Disorders in addition to:]
VLDN (about 125-about 500 μg)+Caffeine (about 25-about 200 mg);
NAC (about 400-about 1200 mg)+Acetaminophen (about 200-about 800 mg)+Caffeine (about 25-about 75 mg);
MSM (about 100-about 1000 mg)+Acetaminophen (about 200-about 1000 mg)+Caffeine (about 25-about 200 mg);
ULDN (about 1-about 125 μg)+Acetaminophen (about 200-about 1000 mg)+Caffeine (about 25-about 200 mg);
VLDN (about 125-about 500 μg)+Acetaminophen (about 200-about 1000 mg)+Caffeine (about 25-about 200 mg);
ULDN (about 1-about 125 μg)+Acetylsalicylic acid (about 75-about 750 mg)+Caffeine (about 25-about 200 mg);
VLDN (about 125-about 500 μg)+Acetylsalicylic acid (about 75-about 750 mg)+Caffeine (about 25-about 200 mg);
MSM (about 100-about 1000 mg)+Acetylsalicylic acid (about 75-about 750 mg)+Caffeine (about 25-about 200 mg);
NAC (about 400-about 1200 mg)+Acetylsalicylic acid (about 75-about 750 mg)+Caffeine (about 25-about 200 mg);
Magnesium Sulfate (about 75-about 1000 mg)+Caffeine (about 25-about 120 mg);
Magnesium Sulfate (about 75-about 1000 mg)+Caffeine (about 25-about 200 mg)+DLPA (about 100-about 1000 mg);

4. Nociceptive Pain

Oral

NAC (about 400-about 1200 mg)+Acetaminophen (about 200-about 1000 mg)+Caffeine (about 25-about 200 mg);
MSM (about 100-about 800 mg)+Acetaminophen (about 200-about 800 mg)+Caffeine (about 25-about 200 mg);
ULDN (about 1-about 125 μg)+Acetaminophen (about 200-about 1000 mg)+Caffeine (about 25-about 200 mg);
VLDN (about 125-about 500 μg)+Acetaminophen (about 200-about 1000 mg)+Caffeine (about 25-about 200 mg);
VLDN (about 125-about 500 μg)+Acetylsalicylic acid (about 75-about 750 mg)+Caffeine (about 25-about 200 mg);
MSM (about 100-about 1200 mg)+Acetylsalicylic acid (about 75-about 750 mg)+Caffeine (about 25-about 200 mg);
NAC (about 400-about 1200 mg)+Acetylsalicylic acid (about 75-about 750 mg)+Caffeine (about 25-about 200 mg);

5. Respiratory Conditions

(e.g., Asthma, COPD, and Neonatal Apnea)

### [Formulations for Emotional and Physical Distress in Addition to:]

Oral

ULDN (about 1-about 125 μg)+Caffeine (about 50-about 250 mg);
VLDN (about 125-about 500 μs)+Caffeine (about 50-about 250 mg);
NAC (about 600-about 1800 mg)+Caffeine (about 50-about 250 mg);
MSM (about 200-about 1200 mg)+Caffeine (about 50-about 250 mg);

Subcutaneous Injection (Dosage Varies by Indication, Time, and Form of Administration)

### Naloxone+Caffeine Citrate;

### Magnesium Sulfate+Caffeine Citrate;

### MSM+Caffeine Citrate;

### NAC+Caffeine Citrate.

### V. Examples

The invention is further described by reference to the following clinical examples, which are provided for illustration only. The invention is not limited to the examples, but rather includes all variations that are evident from the teachings provided herein. Particular materials, dosages, and conditions employed are merely illustrative and not intended to limit the scope of the invention in any way.

All publicly available documents referenced herein, including but not limited to U.S. patents, are specifically incorporated by reference.

### Example 1

The surprising discovery that led to the novel method and composition of the present invention came from a series of clinical trials using drug formulations that were developed by present co-inventor Dr. Stanley M. Crain. Dr. Crain demonstrated that a combination of certain agents, such as rolipram, caffeine, and isobutylmethylxanthine (IBMX), combined with other agents, such as naltrexone and naloxone, resulted in a formulation that could increase mouse tail-flick latencies in a hot-water immersion analgesia study.

The study of Example I was designed and intended to ascertain whether the analgesia (pain relief) demonstrated in the preclinical tail-flick studies could be duplicated in humans. The co-treatment formulations for these studies included combinations of two agents.

Study Design:

For these trials, a hot-water finger-immersion pain paradigm was used, measuring both pain threshold and tolerance. The index finger of the non-dominant hand was submerged in 130° F. water, and the time to the experience of pain (threshold) and the need to withdraw (tolerance) were measured. Baseline scores were compared to those obtained under experimental conditions. Experimental conditions were not blinded for this study.

For the preliminary part of this study, 18 subjects (10 males, 8 females; ages 27-86) were given each of the following single-dose formulations on separate days, to determine the best doses for pain reduction and side-effect profiles:

(1) Caffeine (50 mg)+Magnesium Sulfate (750 mg)

(2) Caffeine (50 mg)+Magnesium Sulfate (5 g)

(3) Caffeine (200 mg)+Magnesium Sulfate (2 g)

(4) Theophylline (100 mg)+ULDN (1 μg)

(5) Theophylline (300 mg)+ULDN (10 μg)

(6) Theophylline (300 mg)+ULDN (100 μg)

(7) Rolipram (1 μg)+ULDN (1 μg)

(8) Rolipram (1 μg)+ULDN (10 μg)

(9) Rolipram (50 μg)+ULDN (100 μg)

(10) Forskolin (200 mg)+Magnesium Sulfate (750 mg)

(11) Forskolin (200 mg)+Magnesium Sulfate (2 g)

These co-treatment formulations generally increased pain thresholds and tolerance as compared to baseline latencies. However, rolipram and forskolin produced undesirable side effects, which led to their elimination in the second phase of the trial. Rolipram, especially at the higher dose (50 produced a queasy sensation for several of the subjects. [One subject took 250 μg of rolipram and experienced nausea, vomiting, and diarrhea. This side-effect profile has been seen in the literature, and has led to rolipram being excluded from clinical development, despite its therapeutic potential.] Forskolin produced mild agitation for several subjects. In addition, the higher doses of magnesium sulfate (2 g, 5 g) produced mild queasy feelings for several subjects, suggesting that the lower dose would be more tolerable for the second phase of the study. Finally, the highest dose of caffeine (200 mg) produced mild agitation for several subjects, and was eliminated from the second phase of the study.

For the remaining conditions, not only was analgesia observed, but also many of the subjects noted a variety of unexpected positive experiences, including a sense of well being and calm, when taking the formulations. However, it was not until the second phase of the study did the clinical importance of these incidental reports become clear.

The same induced pain paradigm was used for the second phase of this study. 60 subjects (38 males, 22 females, ages 18-86), including all of the subjects in the first phase of the study, participated in the study. All subjects were administered each of the following formulations in a single dose, on separate days; while 18 of these subjects (10 males, 8 females) were given these formulations using a twice daily administration over the course of 3 consecutive days. The following formulations were administered to each subject to assess the effect of each agent, as compared to baseline, as well as the synergistic impact of co-treatment:

(1) Caffeine (50 mg)+ULDN (5 μg)

(2) Caffeine (50 mg)+Magnesium Sulfate (750 mg)

(3) Theophylline (300 mg)+ULDN (5 μg)

(4) Theophylline (300 mg)+Magnesium Sulfate (750 mg)

(5) Caffeine (50 mg)

(6) Theophylline (300 mg)

(7) ULDN (5 μg)

(8) Magnesium Sulfate (750 mg)

Two subjects continued to take theophylline (300 mg)+ULDN (5 μg), twice daily, for more than 8 months; a third subject continued to take caffeine (50 mg)+ULDN (5 μg), twice daily, for six months.

As seen in the first phase of the trial, the co-treatment formulations generally increased pain thresholds and tolerance, as compared to baseline latencies. Furthermore, there was generally no analgesic effect when agents were administered alone. In fact, there was a trend toward increased pain sensitivity (hyperalgesia) when caffeine and theophylline were given alone. No side effects were reported for any of the experimental conditions. These findings clearly support Dr. Crain's theory that the combination of these specific drugs produces analgesia.

As noted in the first phase of this study, spontaneous, unsolicited reports from the majority of subjects in these trials revealed an unexpected set of therapeutic benefits from these co-treatment formulations, separate and distinct from the issue of nociceptive pain relief, which was, at the outset, the sole purpose of the study. Completely unplanned and unexpected was the entirely new discovery that these agents, in all co-treatment formulations, dramatically reduced a surprising variety of symptoms in the subjects separate and distinct from nociceptive pain, including gastrointestinal disturbances, emotional and physical agitation, impulsive anger, premature ejaculation, drug cravings, and PMS symptoms. In further studying this phenomenon, another surprising pattern emerged. The formulations were most effective in reducing symptoms (unrelated to the hot water-induced nociceptive pain) in subjects who initially had relatively low pain thresholds and tolerance prior to taking the drugs, suggesting underlying hyperalgesia.

Nearly half the subjects, after taking the co-treatment formulations, indicated that they experienced a positive sense of well-being (positive hedonic tone), in contrast to their accustomed level of stress, anxiety, and irritability. When asked, the others reported that their baseline emotional state was one of a positive feeling of well-being. Thus, in addition to changing physical pain thresholds and tolerance, these formulations demonstrated a remarkable ability to reduce long-standing emotional distress. In fact, two subjects reported an improved ability to control their tendency to respond to events with inappropriate anger and reactivity, giving them a sense of relative peace and self-control.

Another unexpected and surprising finding was seen in the reports of eight subjects who reported that they felt a calming sensation in their gut. They reported that they typically experience discomfort and bloating in their gut, as well as frequent urges to defecate, typical of IBS patients. However, during the days of the trials, they noticed that these gastrointestinal symptoms had remarkably decreased.

Yet another unexpected and surprising finding concerned a female subject who reported that she had been, as was typical for her, experiencing PMS symptoms at the start of the study. Her symptoms included malaise, irritability, and upset stomach. After taking the study formulations, she reported a rapid and dramatic reduction of her PMS symptoms.

One of the more dramatic and surprising findings of the trial was the report by one subject that her long-standing cravings for opioid drugs and alcohol had essentially disappeared, and that she was finally able to completely stop abusing these substances. Needless to say, she is being continued on a combination of theophylline (300 mg) and ultra-low-dose naltrexone (5 μg), twice daily, for maintenance, which has been successful for the past eight months.

Yet another unexpected and surprising finding concerned a male who has asthma and has taken theophylline for over 25 years. Therefore, for the purpose of the study, the only addition to his normal drugs was ultra-low-dose naltrexone (1 μg) taken with each normal dose of theophylline (300 mg SR), twice daily, other than the first day when 100 μg of naltrexone was administered. The subject felt a fairly rapid elimination of symptoms that he had experienced for years, including restlessness, muscular tension, irritability, anger, and anxiety. After a few days, the subject experienced a sense of well-being, contentment, relaxation and calm. These dramatic improvements, emotionally and physically, were sustained throughout a year-long trial, using theophylline (500 mg) with ULDN (5 μg), twice daily. The therapeutic benefits slowly lessened during periods when the naltrexone was withdrawn for a few days and the prior symptoms began to return. However, symptom relief was rapidly restored within hours of adding naltrexone back to the formulation.

Another subject clearly benefited from the combination of caffeine (50 mg) with magnesium sulfate (750 mg). Chronic aches and pains, underlying fears and anxieties, a sense of long-standing dissatisfaction and distress, all were reduced by this treatment. These benefits increased over time, so that subject chose to continue taking the formulation regularly over the following six months. When the formulation was discontinued, the benefits continued with no signs of relapse.

With a common thread of lowered pain thresholds and the benefits of these particular drug combinations, it became evident that these subjects had various forms of Distress Dysfunction, and that a safe and effective treatment for Distress Dysfunction had been discovered for the first time.

Once this discovery was made, a series of retrospective questions were posed to the subjects to assess whether any other benefits occurred. Obviously, this type of inquiry may not be as valid as the independent and spontaneous reports of the subjects during the trial, Nevertheless, this inquiry resulted in confirmation of these symptom relief patterns as well as several other unexpected findings. For instance, these inquiries revealed that two males in the study had experienced significant reduction in premature ejaculation while in the study, a satisfying effect that continued for several days. They both reported a decrease in penile hypersensitivity and increase in ejaculatory control. This finding is consistent with an understanding that premature ejaculation is a manifestation of Distress Dysfunction hypersensitivity.

A consistent pattern among these findings was that symptom reduction remained steady or increased over the course of the twice daily dosing, multi-day trials, suggesting the potential for successful long-term treatment.

One feature of these trials was that the subjects were not aware of whether they were being administered a single agent or combined agents, to determine the potential synergy of cotreatment as well as to reduce placebo effects.

Moreover, another remarkable, unexpected, surprising, synergistic and paradoxical finding concerned the substances used in the study. Theophylline and caffeine are well known for increasing anxiety, restlessness, headaches, and various gastrointestinal symptoms. A number of subjects in these trials complained of these symptoms when given these drugs alone. However, the addition of ultra-low-dose naltrexone or low-dose magnesium sulfate paradoxically reversed the ordinary and expected effect of these drugs, and resulted in a positive, unexpected, dramatic and synergistic effect, reversing patterns of negative, unpleasant effects normally associated with these drugs when administered alone. Not only were complaints of side effects and hyperalgesia eliminated in the co-treatment condition, but, as reported above, subjects experienced a dramatic improvement in positive hedonic tone, which included a significant reduction in symptoms that they had been experiencing prior to the study. At the same time the low-dose naltrexone and magnesium sulfate, alone, had neither benefit nor side effects. Not only did these findings confirm the unexpected synergistic therapeutic effects of these co-treatment formulations, but they also suggested a completely novel method for reducing the typical side effects of these medications.

### Example 2

A preliminary study of Distress Dysfunction included four patients in psychotherapy, all of whom suffered from years of Distress Dysfunction, characterized by chronic emotional distress, anxiety, irritability, and anhedonia, which were not improved by traditional psychiatric medication or years of psychotherapy.

The four patients (2 males, 2 females; ages 28-56) took a twice-daily formulation of caffeine pills (50 mg) and magnesium sulfate solution (500 mg), which, within two days, produced meaningful relief from these chronic symptoms. The patients reported a significant reduction in anxiety, irritability, and depression, and an increase in satisfaction and well being. This rapid relief of chronic intractable symptoms was remarkable. However, when it became clear that one patient had dramatically greater improvement than the others, an assessment revealed that he regularly consumed acetylsalicylic acid and acetaminophen to help him sleep.

Theorizing that these agents were having a dramatic and synergistic interaction with the tested formulation, the other three patients agreed to add one of these analgesics as well. All patients continued to take the caffeine and magnesium sulfate, and added acetaminophen (500 mg, twice daily). The results showed an increased improvement in all four patients. As one of these patients described the effect of this enhanced formulation, “for the first time in my life I feel normal, like I have a thicker skin.”

More recently, these patients were given a consistent twice-daily formulation of acetaminophen (500 mg), caffeine (65 mg), and naltrexone (1 μg). This formulation has produced very reliable relief of chronic emotional distress for the past twelve months with no side effects.

Given these unexpected, dramatic findings, a 42 year old female patient, suffering from Distress Dysfunction characterized by chronic alcohol and opioid cravings and abuse over many years, was given this formulation. Much to her surprise, the simple combination of acetaminophen, (500 mg), caffeine, (65 mg), and very-low-dose naltrexone (50 μg), given three times daily, completely eliminated her alcohol and drug cravings as well as any substance use. Years of psychotherapy and traditional medications had little or no impact on these serious symptoms. This treatment has been completely successful for the past ten months. To test the efficacy of the formula, without the patient's awareness, the naltrexone was replaced by a placebo for one week. Within a few days, cravings returned and the patient was tempted to relapse on several occasions. The full formula was continued after the week, and the patient's cravings were, once again, completely eliminated.

### Example 3

To assess the efficacy of this innovative treatment for Distress Dysfunction with a very serious, chronic disorder that is typically considered to be “untreatable”, a 28 year old female, diagnosed with Autistic Spectrum Disorder, was treated with ultra-low-dose naltrexone and caffeine.

Relevant symptoms included the following: limited eye contact, avoidance of even minor stimulation or frustration, highly reactive emotionally and physically to minor frustrations, extremely irritable and quick to anger, fearful of any arousal (both positive and negative), isolation and anhedonia. The subject historically avoided caffeine as well as physical exertion and excitement of any kind, since she felt that caffeine and these activities tended to make her intensely agitated. Despite this chronic caffeine reaction, the treatment consisted of ULDN (5 μg daily) and caffeine (150 mg daily). Therapeutic benefits were evident within the first few days of treatment, and steadily increased over the next few weeks.

Paradoxically, for the first time in the patient's life, she experienced positive hedonic tone (a positive sense of well-being, happiness, pleasure and contentment): paradoxically, she felt a calming as well as an energizing response from the caffeine with none of the agitation she had always experienced in the past. Moreover, the patient began to realize that her normal reactivity to even minor stresses disappeared. She felt “normal” for the first time in her life, able to experience some simple pleasures, including a budding enjoyment of spending time with others, and even laughing with them. As she put it, “I don't have to keep myself in an emotional straightjacket for fear of getting all worked up and losing control.” Treatment has continued for the past six months, with the therapeutic benefits maintained. Recently, she has been able to enter a successful romantic relationship for the first time in her life.

This novel treatment of a patient with Autistic Spectrum Disorder is a striking example of the power of this co-treatment paradigm. The patient had been in treatment of various types, including psychiatric medications, for most of her life, with no meaningful improvement. Within a few days of taking the described formulation, her life-long chronic condition began to improve for the first time in her life. This finding supports our conclusion that this treatment can resolve chronic Distress Dysfunction symptoms and disorders with relatively innocuous agents. This case also specifically reveals the relevance of this formulation for the treatment of autism. The paradoxical caffeine reaction supports our conceptual understanding of the role of, and the ability to restore healthy functioning to the opioid and related neurotransmitter systems.

### Example 4

In conducting these trials, it became clear that certain subjects had a markedly greater increase in pain thresholds and pain tolerance when given a co-treatment of a Receptor Switcher with Endorphin and Synergistic Enhancers, as compared to the more typical response of either given alone. Upon questioning the subjects, a completely unexpected discovery was made. Most of these “outliers” were, coincidentally, taking some form of exogenous non-opioid analgesic, such as acetaminophen, ibuprofen, or “baby aspirin.” (The investigators had not screened for non-prescription drug usage prior to study participation.) What was particularly surprising and unexpected was that the subjects who took an exogenous non-opioid analgesic during the study, did not, on average, have higher baseline pain threshold/tolerance scores than subjects who were not. Therefore, there was clear evidence of a marked synergistic combination of the exogenous non-opioid analgesic with the Receptor Switcher, since this produced the greatest analgesic response.

To confirm this finding, the same pain threshold/tolerance hot-water finger-immersion paradigm was used with five subjects who were given, during separate testing sessions: (1) acetaminophen (500 mg) alone; (2) theophylline (300 mg) combined with ultra low dose naltrexone (1 μg); and (3) (1) and (2) combined. The increase in pain threshold/tolerance (over baseline), in order of greatest pain relief, was (3), then (2) then (1). Moreover, a dramatic synergy was uncovered since the increase in pain tolerance scores for (3) was greater than the sum of (1)+(2). In fact, the same results were found when using “baby aspirin” (81 mg) instead of acetaminophen. Although the “baby aspirin” showed no change in pain threshold when administered alone, surprisingly and unexpectedly, the combined formulation produced the most dramatic analgesia, again demonstrating the synergistic potentiation of the exogenous non-opioid analgesic using this formula.

These findings were dramatic and completely unexpected since there had been no prior teaching regarding the ability of a Receptor Switcher and an Endorphin Enhancer to potentiate analgesia produced by an exogenous non-opioid analgesic; nor had there been any teaching to suggest combining either a Receptor Switcher or an Endorphin Enhancer with an exogenous non-opioid analgesic to enhance its pain-relieving effects. The implications are significant. For the first time, there is evidence to suggest that an enhanced non-opioid formulation might be sufficiently potent for moderate-to-severe pain. Although it is generally accepted that exogenous opioid drugs create analgesia through their impact on the endogenous opioid system, there is no well-accepted understanding of how exogenous non-opioid analgesics actually work. There has been, however, some speculation that the analgesic effect of NSAIDs (Pernia-Andrade et al., Eur. J. of Pharmacology, 111(1):19 about 1-about 200 (2004)) and acetaminophen (Raffa & Walker, Eur. J. of Pharmacology, 503(1-2):209-210 (2004)) involve opioidergic mechanisms.

### Example 5

To determine the relative benefits of administering agents alone as compared to their combined synergistic benefits, a trial was conducted over a 10 month period with 19 patients in psychotherapy suffering from various Distress Dysfunction conditions, including anxiety, obsessive-compulsive disorders, depression, drug and alcohol addictions, behavioral addictions, psychogenic and neuropathic pain, eating disorders, and sexual dysfunctions.

Without identifying the agents, each patient was given one of the following agents, once daily, for a period of two weeks: ultra-low-dose naltrexone (5 μg), very-low-dose roflumilast (5 μg), caffeine (65 mg), and acetaminophen (500 mg). Over the course of 8 weeks, each patient was administered all of the individual agents. During the next 8 weeks, each patient rotated through the following formulations, each given for a 2 week period:

(1) ULDN (5 μg)+Roflumilast (5 μg)

(2) ULDN (5 μg)+Caffeine (65 mg)

(3) ULDN (5 μg)+Acetaminophen (500 mg)

(4) ULDN (5 μg)+Caffeine (65 mg)+Acetaminophen (500 mg)

The results of this initial 4 month phase of the study were remarkably compelling and supported the critical importance of combining a Receptor Switcher (e.g., ULDN) with an Endorphin Enhancer (e.g., roflumilast or caffeine) and/or a Synergistic Enhancer (e.g., acetaminophen). When roflumilast, caffeine, or acetaminophen was administered alone, no therapeutic benefits were reported or observed, and at least half of the patients reported some degree of increased distress symptoms. When ULDN was combined with any of the other agents, a wide variety of Distress Dysfunction symptoms were reduced within the first 1-2 days, including anxiety, obsessive thoughts, compulsive behaviors, cravings, alcohol abuse, as well as aches and pains. Of these co-treatment formulations, the combination of ULDN, Caffeine, and Acetaminophen seemed to be generally the most effective. These benefits generally increased over the two week treatment period.

However, a very unexpected and surprising finding occurred when administering the ULDN alone. While nearly half of the patients experienced no effect from ULDN alone, 10 patients had dramatic improvement in their symptoms simply by the taking the naltrexone. Long-standing symptoms that were significantly reduced and/or eliminated included depression, anxiety, drug and food cravings, anger and irritability, emotional hypersensitivity and reactivity, aches and pains, as well as general malaise and agitation. Instead, for the first time in years, these patients felt a sense of normalcy and well-being, without their normal feelings of distress and unease.

Inquiry into the differences between this subset of patients and the others revealed a very clear, yet surprising pattern—therapeutic benefits occurred for patients that were on SSRI and SNRI medications, including Lexapro, Prozac, Paxil, and Effexor. Suddenly, and unexpectedly, it became clear that ULDN was able to combine in a synergistic way with an SSRI or SNRI to dramatically potentiate their therapeutic benefits in reducing chronic Distress Dysfunction symptoms, which had not significantly improved despite several years of psychotherapy and medication. Furthermore, when ULDN, a Receptor Switcher, was combined with Endorphin Enhancers (caffeine or roflumilast), and added to the patient's ongoing SSRI or SNRI, (Synergistic Enhancers), maximal therapeutic benefits were seen, especially with the most chronic and resistant symptoms of depression, anxiety, and addictions. Clearly, completely novel formulations had been discovered, which led to, and validated, the novel principles for treating Distress Dysfunction that define this invention.

Given these remarkable and unexpected findings, the focus of the remaining six months of the clinical study shifted to maintaining each patient on the formulation that worked best for their symptoms. All patients already on an SSRI or SNRI continued on this medication in combination with ULDN. Five of these patients also continued to take caffeine to maximize relief from emotional distress symptoms. Two of the patients not taking an SSRI or SNRI prior to the study were prescribed one to add to the ULDN treatment, which contributed to their clinical improvement. The remaining seven patients were maintained effectively on the combination of ULDN and caffeine. Acetaminophen was used effectively by four pain patients on an “as needed” basis for breakthrough pain during this phase of the study.

### Example 6

To test the effects of an ultra-low dose of a selective cAMP PDE4 inhibitor and a Receptor Switcher on Distress Dysfunction symptoms, eight subjects were given ultra-low-dose roflumilast (1 μg) with ultra-low-dose naltrexone (1 μg), once daily, for a period of four weeks. All subjects suffered from long-standing problems with catastrophic worries, emotional and physical tension, distressing pain, and low-grade depression. Roflumilast was chosen given its potential potency and safety profile, as compared to theophylline, and reduced side effects, as compared to rolipram.

All four subjects reported similar benefits from the co-treatment formulation within the first two days of initiating the treatment. Each of them independently described that they felt an increased calm, a greater sense of general “well being” and a decrease in worries and stresses. They all indicated that they were less emotionally reactive to minor stresses, yet more able than usual to respond appropriately to situations without being distracted by their usual exaggerated “catastrophic” emotional and cognitive overreactions. They also noted that they had less of their normal aches and pains, particularly for an individual with mild arthritis who felt a much greater sense of mobility in addition to a reduction in pain. Moreover, all of them noted that they did not feel any type of “drugged state” or “high” of any kind, just a sense of normalcy. One subject noted that he had a very mild headache during the trial, but that it went away by the end of the fourth day. All therapeutic benefits consistently improved over the two-month trial.

What was remarkable about this small trial was the remarkably ultra-low-doses of both agents—one microgram of each agent, twice daily. Roflumilast is conventionally used at 500 micrograms for COPD, and naltrexone is used at 50 milligrams for addiction. Therefore, these doses are 1/500^{th} to 1/50,000^{th} of the normal doses of these agents. Yet, this novel cotreatment formulation had significant effects in reducing symptoms of Distress Dysfunction, regarding emotional and cognitive anxieties and worries as well as aches and pains of conditions and mobility problems, such as arthritis. What was also remarkable, with the exception of one mild headache that resolved within days and did not return during the 2 month trial, was that these low doses produced no side effects or signs of withdrawal or any altered cognitive or emotional states, other than a sense of normal well being. Thus, this simple formulation was able to produce positive emotional and physical hedonic homeostasis.

### Example 7

Six subjects with asthma, already taking theophylline (300-600 mg daily) for many years, were given ultra-low-dose naltrexone (5 μg, once daily) over periods ranging from one month to a year.

Remarkably and unexpectedly, these subjects experienced a dramatic reduction in a variety of side effects from the medication, including hyperalgesia, agitation, anxiety, and various gastrointestinal symptoms, within the first 2-3 days of adding ULDN. Sensitivity to pain was greatly reduced, including chest pain associated with asthma.

Three subjects were switched from theophylline to roflumilast (250 mg, once daily). Roflumilast alone, like theophylline, is known to produce side effects, such as increased pain sensitivity, anxiety, and GI symptoms. However, when cotreated with ultra-low-dose naltrexone (5 μg, once daily), these side effects were eliminated. Furthermore, given the increased calming influence and pain relief from the cotreatment formula, it clearly provided a more effective treatment for asthma.

This finding with asthma sufferers suggests that this increased therapeutic efficacy and safety would also occur when using this cotreatment formula for COPD patients.

More recently, the three subjects were transitioned to roflumilast (250 mg, once daily) and n-acetyl-cysteine (600 mg, twice daily). The results have been even more compelling, especially over the three-month trial. Asthma has been consistently controlled, with an increased sense of calm and well being, and an absence of typical theophylline and roflumilast side effects. A clear synergistic effect has been discovered between these two agents, both regarding their respiratory benefits, but also their ability to resolve symptoms of Distress Dysfunction that are a function of the asthmatic condition as well as a side effect of the medications. Therefore, a novel treatment for asthma and COPD has been discovered that combines a PDE inhibitor, known for its therapeutic benefits for respiratory conditions, and a Receptor Switcher, which blocks the noxious side-effects of the PDE inhibitor and increases a sense of well being and calm.

### Example 8

### Summary of Validation Studies

In summary, a series of studies have been conducted to validate the science and formulations contained in this application. This research has consisted of two primary forms of investigation: induced pain and clinical case studies.

A. Induced Pain Trials

More than 100 subjects have participated in hot and cold water induced pain trials, dramatically supporting the principles and formulations discovered by this invention for the safe and effective relief of pain. In addition to the hot-water finger immersion studies described above, two Jeio Tech cold-pressor testing units were purchased since this methodology represents the state-of-the art in induced pain research. Consensus in the research literature, as well as the findings described herein, suggest that over-the-counter analgesics, such as acetaminophen, aspirin, and ibuprofen, have no greater analgesic effect than placebo in cold-pressor pain tolerance testing. Only exogenous opioid drugs, such as oxycodone, have been shown to have a significant analgesic effect, making the cold-pressor the “gold standard” for moderate-to-severe pain assessment.

Following several preliminary multi-site trials using a wide variety of agents, doses, and formulations, a cold-pressor induced pain study was conducted in order to determine the impact of specific agents on the experience of pain threshold and tolerance. Healthy volunteers were recruited who were not taking any relevant medications or supplements, resulting in a total of 12 subjects (7 men, 5 women; ages 18-86). During the initial phase of this study, the following agents and doses were administered to subjects and pain threshold and tolerance latencies were assessed. Receptor Switchers were ULDN (5 μg), NAC (600 mg), and MSM (500 mg). Endorphin Enhancers were roflumilast (50 μg), ginkgo biloba (120 mg), glutamic acid (50 mg), and DLPA (250 mg). Exogenous Opioids were tramadol (5, 10 mg), hydrocodone (5 mg)/acetaminophen (500 mg), and oxycodone (2.5, 5 mg). Synergistic Enhancers were acetaminophen (500 mg), aspirin (300 mg), and white willow bark (400 mg). Subjects were blinded as to each condition. Active agents were compared to each subject's baseline and placebo latencies, using a within-subject design.

As predicted, the only individual agents that produced a clear trend toward increased pain threshold and tolerance latencies, as compared to baseline and placebo scores, were Exogenous Opioids; moreover, only oxycodone (5 mg) consistently showed this effect. Tramadol, lower dose oxycodone, hydrocodone/acetaminophen, and the non-opioid analgesics did not generally exhibit analgesia on the cold pressor. This finding is consistent with prior studies in the literature. When administered alone, none of the experimental agents showed any trend toward pain relief; instead, roflumilast and glutamic acid showed clear hyperalgesic effects when administered alone. Therefore, if co-treatment formulations were to show analgesic effects, dramatic synergy with the Receptor Switcher would be necessary. Regarding side effects, all of the opioid drugs produced classic narcotic side effects including nausea, light-headedness, dizziness, a feeling of being “high,” and a desire to obtain more of the drug. None of the other agents exhibited such effects, including the non-opioid analgesics.

For the second phase of this cold pressor induced pain study, the subjects were given a variety of co-treatment formulations as well as a placebo. Formulations were selected based upon results in previous testing, side effect and known safety profiles, and marketing potential. The subjects were blinded to the specific agents administered. For each testing session, each subject was administered one of the following formulations:

(1) ULDN (5+Roflumilast (50 μg)

(2) NAC (600 mg)+Ginkgo Biloba (120 mg)

(3) NAC (600 mg)+Glutamic Acid (50 mg)

(4) MSM (500 mg)+Ginkgo Biloba (120 mg)

(5) MSM (500 mg)+Glutamic Acid (50 mg)

(6) NAC (600 mg)+Ginkgo Biloba (120 mg)+DLPA (250 mg)

(7) NAC (600 mg)+Ginkgo Biloba (120 mg)+WWB (400 mg)

(8) NAC (600 mg)+Ginkgo Biloba (120 mg)+Tramadol (5 mg)

(9) NAC (600 mg)+Ginkgo Biloba (120 mg)+Oxycodone (2.5 mg)

(10) ULDN (5 μg)+Roflumilast (50 μg)+Tramadol (5 mg)

(11) ULDN (5 μg)+Roflumilast (50 μg)+Oxycodone (2.5 mg)

In addition to the single-dose administrations, 6 of these subjects (4 men, 2 women) participated in a twice-daily dosing study over seven consecutive days. Cold pressor testing was done during the first, third and seventh day. The following formulations were used for this multi-day trial:

(1) ULDN (5 μg)+Roflumilast (50 μg)

(2) NAC (600 mg)+Gingko Biloba (120 mg)

(3) NAC (600 mg)+Ginkgo Biloba (120 mg)+WWB (400 mg)

When administered alone, these agents were shown to be no better than placebo. In fact, many of the Endorphin Enhancers, such as roflumilast, theophylline, caffeine, and glutamic acid, when given alone, tended to result in shorter pain tolerance latencies, indicating hyperalgesic effects. In contrast, when a Receptor Switcher (ULDN, NAC, or MSM) was combined with an Endorphin Enhancer (roflumilast, ginkgo biloba, or glutamic acid), these formulations were generally more effective in producing analgesia than placebo and non-opioid analgesics (including acetaminophen, ibuprophen and aspirin, which were no more effective than placebo in the cold pressor paradigm). The combination of ULDN and roflumilast was slightly more effective in increasing pain tolerance for most subjects. Moreover, the addition of a third agent to these formulations, white willow bark or DLPA, potentiated analgesia to the level of oxycodone (5 mg). Finally the addition of a subclinical dose of tramadol (5 mg) or oxycodone (2.5 mg) in combination with a Receptor Switcher and Endorphin Enhancer produced analgesic effects that were generally more potent than 5 mg of oxycodone administered alone. This synergistic effect was particularly pronounced when the very low dose Exogenous Opioid was added to ULDN and roflumilast. All of these results have been consistent for both single dose and 7-day, twice daily dosing, with a clear tendency toward increased and more consistent pain relief with repeated dosing over the course of one week.

Therefore, these remarkably safe, novel non-opioid formulations, simply combining a Receptor Switcher, such as ULDN, NAC, or MSM, with an Endorphin Enhancer, such as roflumilast, ginkgo biloba, or glutamic acid, were able to reduce moderate-to-severe pain more effectively than conventional NSAIDs. Furthermore, the addition of a NSAID, such as white willow bark, or a second Endorphin Enhancer, such as DLPA, created a formulation that was generally as effective as an Exogenous Opioid, without the noxious side effects and safety issues, such as tolerance and dependence, of narcotic drugs. Alternatively, the addition of 5 mg of tramadol a less regulated “weak” opioid (10 mg of tramadol is equipotent to 1 mg oxycodone), created a formulation that was potentially more effective than 5 mg of oxycodone. Obviously, there are enormous clinical benefits to have discovered, for the first time in clinical trials, remarkably safe formulations that can either replace exogenous opioids or potentiate remarkably low doses of both weak (tramadol) and powerful (oxycodone) opioids for the relief of moderate-to-severe pain.

Subjects in these induced pain trials consistently reported an absence of side effects using the formulations (in contrast to the typical side effect profile reported when conventional dose exogenous opioids were administered, including constipation, itching, emotional and cognitive disorientation, and yearnings to take more of the opioid). Normal acute reflexive pain was always maintained, with no changes in cognitive abilities (again, in contrast to the “conventional dose” opioids, often characterized by blurred judgment and logic, and interference with adaptive acute reflexive pain). Moreover, subjects frequently reported many remarkable therapeutic benefits, consistent with a reduction in Distress Dysfunction, including an increased sense of calm and well being, and a significant decrease in anxiety, worries, obsessions, anger, irritability, distractibility, cravings, GI symptoms, and general aches and pains. Five subjects in this study with chronic back, shoulder, and arm pain reported remarkable relief during the study, particularly when using co-treatment formulations that included white willow bark and very low dose opioids. Many of the subjects in the study chose to continue a non-opioid, non-prescription formulation on their own, preferring them to any conventional pain and anxiety medications currently on the market. Therefore, these novel formulations were able to simultaneously reduce emotional and physical distress and pain, with no side effects. While similar in pain relief, the cognitive and emotional differences between the formulations of the invention and exogenous opioids were quite remarkable.

B. Clinical Case Studies

More than 100 outpatient psychotherapy patients with various forms of Distress Dysfunction have participated in clinical case studies using the formulations of the invention, from one month to more than one year. Patients were selected to be in included in this project who had moderate-to-severe emotional and physical distress disorders, which were not effectively being treated by conventional therapies including psychotherapy and prescription medications, such as anti-depressant medication (e.g., SSRIs, SNRIs), anti-anxiety medication (e.g., benzodiazepines), and pain medication (e.g., opioids, NSAIDs). Therefore, the disorders presented by these patients were particularly challenging and resistant to conventional treatment. Many of these patients (45) suffered from emotional distress, including anxiety, obsessive-compulsive symptoms, panic, social fears, depression, dysthymia, anger, irritability, and emotional agitation and outbursts. Another group of patients (21) suffered from pain, including fibromyalgia, neuropathic pain, arthritis, headaches as well as back, shoulder, and neck pain. Many of these patients also suffered from anxiety and depression. Another group of patients suffered from alcohol (13) and opioid (4) dependence, which lasted for several years with daily abuse. Another group of patients (8) suffered from eating disorders. Another group (7) suffered from IBS, primarily diarrheal. Finally, 4 patients were diagnosed with adult Autistic Spectrum Disorder, particularly Asperger's Syndrome. A number of these patients also had co-existing problems, including sexual dysfunction, behavioral addictions, premenstrual syndrome, seasonal affective disorder, and social and relationship conflicts.

All treatment was unblinded, and explained as novel formulations for Distress Dysfunction. Patients were administered formulations that included at least one Receptor Switcher, including ULDN (5 or 125 μg, once daily), NAC (600 mg, twice daily), MSM (250 mg, twice daily), and magnesium sulfate (250 mg, twice daily). Most formulations also included at least one Endorphin Enhancer including ginkgo biloba (120 mg, twice daily), caffeine (50 mg, twice daily), glutamic acid or MSG (50 mg, twice daily), and DLPA (250 mg, twice daily). Synergistic Enhancers were often included in the formulations, particularly SSRI and SNRI medications that the patients were typically taking prior to adding the experimental formulations (about 25% of patients). For those patients already on an SSRI or SNRI, the following daily doses were used, once the patient was stabilized on the co-treatment formulation: escitalopram (10 mg, once daily), fluoxetine (20 mg, once daily), citalopram (20 mg, once daily), or venlafaxine (75 mg, once daily). For most patients, this was either the same or lower dose than previously prescribed. If the patient was not already on an SSRI or SNRI, one or more Synergistic Enhancers were added to the formulation for more serious conditions, including 5HTP (50 mg, twice daily), SAMe (200 mg, once daily), PharmaGABA (200 mg, once daily), and tyrosine (500 mg, once daily) (about 18% of patients). Ten pain patients began the treatment program using a prescription narcotic pain medication. Once stabilized on the cotreatment formulation, patients either were able to completely withdraw from these drugs, or were able to maintain a relatively low dose of the opioid medication: tramadol (10 mg, twice daily) or oxycodone (5 mg, twice daily). As an alternative to narcotic pain medications, NSAIDs, especially white willow bark (250 mg, twice daily) were added to the formulation (about 10% of patients). Nearly all of the patients were given more than one formulation over time to assess the differential benefits, using intra-subject comparisons, as well as to maximize therapeutic effects.

The most recent formulations that were used for each patient, which maximized symptom reduction, are listed below. The formulations are categorized by clinical indication, with the number of patients taking the specific formulation indicated. Specific agent doses for each agent in the formulation are indicated above.

TABLE 1
Emotional Distress (Anxiety, Depression, Anger)

| # Patients | Formulation |
|---|---|
| 9 | NAC + Ginkgo Biloba |
| 9 | NAC + Ginkgo Biloba + 5-HTP |
| 7 | NAC + Ginkgo Biloba + 5-HTP + PharmaGABA |
| 3 | NAC + Ginkgo Biloba + Escitalopram |
| 3 | NAC + Ginkgo Biloba + Venlafaxine |
| 3 | ULDN + Fluoxetine |
| 3 | ULDN + Escitalopram |
| 3 | ULDN + Ginkgo Biloba + Escitalopram |
| 3 | MSM + Caffeine + Citalopram |
| 3 | NAC + Escitalopram |
| 3 | NAC + Ginkgo Biloba + PharmaGABA |

TABLE 2
Physical Distress (Pain)

| # Patients | Formulation |
|---|---|
| 4 | MSM + Ginkgo Biloba |
| 4 | MSM + Ginkgo Biloba + White Willow Bark |
| 4 | NAC + Ginkgo Biloba + White Willow Bark |
| 3 | MSM + Glutamic Acid + White Willow Bark + DLPA |
| 2 | NAC + Glutamic Acid + White Willow Bark + DLPA |
| 2 | NAC + Ginkgo Biloba + Tramadol |
| 2 | ULDN + Glutamic Acid + Oxycodone |
| 2 | ULDN + Ginkgo Biloba + Escitalopram |

TABLE 3
Addiction (Alcohol & Drug Dependence,
Behavioral Addictions, Cravings)

| # Patients | Formulation |
|---|---|
| 4 | NAC + Ginkgo Biloba + Escitalopram |
| 4 | NAC + Ginkgo Biloba + GABA + Tryosine |
| 4 | NAC + Ginkgo Biloba + Glutamic Acid + 5-HTP |
| 3 | NAC + Ginkgo Biloba + GABA |
| 3 | NAC + Ginkgo Biloba + SAME-e |
| 3 | ULDN + Ginkgo Biloba + Escitalopram + Tryosine |
| 3 | NAC + Glutamic Acid + White Willow Bark |

TABLE 4
Gastrointestinal (IBS, PMS, Crohn's)

| # Patients | Formulation |
|---|---|
| 2 | NAC + Ginkgo Biloba |
| 2 | Magnesium Sulfate + Ginkgo Biloba |
| 2 | Magnesium Sulfate + Ginkgo Biloba + GABA |
| 2 | ULDN + Gingko Biloba |

TABLE 5
Autistic Spectrum

| # Patients | Formulation |
|---|---|
| 2 | NAC + Caffeine + GABA |
| 2 | NAC + Ginkgo Biloba + Magnesium |

Although placebo factors must always be considered, the results of these case studies were, nevertheless, remarkable, especially given the limited success of conventional treatments with these patients. All patients reported clear, and at times dramatic, improvements in their emotional and physical distress, including pain and GI symptoms. For many patients, the initial formulation was effective; for others, modifying the formula, including adding Synergistic Enhancers, was needed to achieve maximal benefits. Regardless of the Distress Dysfunction indication, therapeutic benefits of this novel treatment were only observed when at least one Receptor Switcher was included in the formulation. All of these patients had been suffering from significant symptoms for over a year, and many for more than a decade. For those on an SSRI or SNRI and/or in psychotherapy, prior benefits were compared to those obtained with the addition of our novel cotreatment formulations. Furthermore, many of the patients had previously taken many of the agents used in these formulations, such as ginkgo biloba, caffeine, 5HTP, GABA, SSRIs, SNRIs, and NSAIDs, with either limited or no relief, and often with undesirable side effects. The key for all patients in these case studies is that most had never taken any of the Receptor Switchers, such as ULDN, NAC, MSM, or magnesium sulfate, and none had ever taken these agents in combination with the Endorphin and/or Synergistic Enhancers used in these formulations.

The most reliable therapeutic benefit these novel formulations provided for these patients was a dramatic reduction in emotional distress, particularly a remarkable decrease in anxiety, irrational fears and worries, panic, obsessions, compulsions, anger and irritability, and depression. Increased attention, concentration, productivity, and overall social and emotional functioning were consistently reported. Nearly all patients experienced a greater calm and sense of well being than they felt in years, a benefit that was maintained consistently during the entire course of treatment. One 87-year-old female, with severe arthritis, chronic worries, and depression, remarked that “I felt for the first time in years that life is worth living.”

Eight patients with obsessive-compulsive symptoms that were not controlled by psychotherapy or medication, including SSRIs and SNRIs, experienced a dramatic reduction in the symptoms that had previously controlled their lives. SSRI treatment was clearly enhanced using Receptor Switchers and Endorphin Enhancers. The addition of Synergistic Enhancers, particularly GABA and 5HTP, also increased therapeutic benefits. IBS symptoms were reduced for the first time in years for seven patients. Eating disorder symptoms (that had remained unchanged despite years of psychotherapy and conventional medication) were remarkably reduced, including a decrease in food cravings, binging, purging, and general sense of emptiness often experienced by many of these patients. Increased sexual functioning, including a reduction in premature ejaculation was reported by several patients. Four patients with Asperger's Syndrome were able to relax and become more social, as well as less irritable with a great reduction in emotional outbursts. All 17 patients with alcohol and opioid dependence were able to completely eliminate their substance abuse after years of unsuccessful treatment, and also experienced a greater sense of well being and reduction in cravings and anxieties.

Overall, physical distress was dramatically reduced, with benefits increasing significantly over time. Complaints about moderate-to-severe pain, regardless of the etiology, were generally reduced, without use of opioid analgesics. No tolerance was ever experienced, and the formulations maintained their effectiveness throughout all case studies. For nearly all patients, continued improvement was seen over time. No side effects were reported, with all cognitive and emotional functions improved, in contrast to the experience reported by patients when they have used conventional medications, especially exogenous opioid drugs and traditional anti-anxiety medications. Cognitive and emotional functions were significantly improved, with greater clarity, judgment, attention, mood, and motivation.

Therefore, while placebo factors and concurrent psychotherapy were potential confounding factors, these case studies offer compelling evidence of meaningful therapeutic benefits for a wide variety of Dysfunctional Distress disorders, conditions, and symptoms, using these remarkably safe pharmaceutical formulations. Specifically, these case studies, which always included a Receptor Switcher (ULDN, NAC, MSM and/or magnesium sulfate) combined with an Endorphin Enhancer (ginkgo biloba, caffeine, glutamic acid, and/or DLPA) and/or a Synergistic Enhancer (SSRI, SNRI, white willow bark, 5HTP, SAMe, pharmaGABA, tryosine), provide validation for the remarkable safety and clinical effectiveness of these pharmaceutical formulations.

While both dosing levels of naltrexone were generally effective, the benefits from using the relatively higher dose (125 micrograms) were more consistent within and among patients. Therefore, these clinical case studies support the conclusion that greater efficacy and reliability is produced, over all populations and indications, when using VLDN in the 100-150 microgram range. Variable bioavailabilty of oral naltrexone among individuals probably accounts for the need for this dosing range to produce the most consistent therapeutic effects. Clearly, more trials will be needed to determine the moste effective dosing of all agents in these formulations for different populations and indications.

A striking aspect of these case studies was the remarkable safety and effectiveness of “grandfathered nutraceuticals.” These active agents are generally regarded as safe (GRAS) and were available for purchase in the United States prior to 1994, and, therefore, are regulated by the FTC for ‘truth in marketing’ claims rather than the FDA. These relatively safe agents have been shown to be effective, in the correct combination, for patients with serious emotional and physical distress disorders. No benzodiazepines were used throughout this treatment program, which is unusual given the extent of anxiety presented by these patients. At the onset of the program, 10 pain patients were taking prescribed narcotic opioid medications that were ineffective, including Vicodin and Oxycontin. Six of these patients discontinued these opioid medications altogether, once the novel formulations were begun, and the remaining 4 patients were able to lower their opioid drugs significantly as part of the overall formulation. In addition, the 4 patients who began the program addicted to, and abusing, narcotic opioid drugs (that were not taken specifically for pain), were able to discontinue these drugs altogether, once they began taking the novel formulation. Finally, while the 30 patients who began the program on anti-depressant medication generally continued on it as part of their overall formulation, they were able to maintain or lower the dose and yet experience remarkable reductions in symptoms of emotional and physical distress. Most dramatically, 72 patients participating in this program (over 70% of all cases) were able to experience remarkable reduction in chronic symptoms of emotional and physical distress, including pain and addiction, using only grandfathered nutraceutical formulations, which included NAC, MSM, ginkgo biloba, glutamic acid, caffeine, white willow bark, 5HTP, GABA, and SAMe. These agents have been used for decades, are well-known to be relatively safe for long-term use, and have very clear health benefits for the immune, circulatory, respiratory, and other systems of the body.

In sum, a series of clinical trials and case studies have been conducted which surprisingly and dramatically support the emotional and physical health restoring benefits of these novel formulations. Over 200 subjects and patients have been given one or more of these novel formulations. There have been virtually no side effects or any adverse events observed in any of these individuals. In addition, all reports indicate that these formulations do not produce any feeling of “taking a drug,” in contrast to the “drugged” experience typically seen in most medications currently available for the treatment of symptoms of Distress Dysfunction, such as tranquillizers and opioid drugs. At the same time, the therapeutic benefits of these novel formulations have been extremely positive, and dramatically more effective that any of the agents when taken alone, or in combinations within each functional category of agents, thereby validating the surprising synergistic healing power of this invention's unique formulations. Therefore, evidence indicates that the addition of Reeptor Switchers creates synergistic therapeutic benefits well beyond the reduction of tolerance, since the benefits are remarkably better than the administration of the agents or drugs when taken alone, from the very start of treatment.

Using this invention's principles, new formulations, using different agents with similar functional properties, have reliably been effective, validating these principles and the entire invention. Furthermore, since these formulations clearly are safer and more effective than conventional treatment, they provide validation for both the existence of Distress Dysfunction, as defined in this patent, and for a revolutionary group of pharmaceutical formulations, which restore healthy homeostatic balance to the neurotransmitter systems within the nervous system.

### Example 9

A revealing clinical example of the relative effectiveness of these novel formulations for Distress Dysfunction is reflected in a case study of a professional couple, married for over 20 years. They both suffered from chronic alcoholism and tobacco use for most of their marriage, and the husband regularly abused a variety of other drugs, including marijuana, tranquillizers, and narcotic pain medications. Despite several hospitalizations for toxic effects of chronic alcohol abuse, neither partner was able to reduce their alcohol use. Both spouses also suffered from moderate depression and anxiety; while the husband had episodic panic attacks and periods of severe depression. The husband often had angry outbursts that were considered abusive and often went on for hours and even days. Each spouse had been in individual psychotherapy for years, and the couple has been in marital therapy for the past three years. The husband has been taking a variety of SSRI medications and anti-anxiety medications for years. None of these treatment approaches were effective.

Initially, the husband was given ultra-low-dose naltrexone (5 micrograms daily) in addition to continuing escitalopram. For the first time, with the addition of ULDN, he was able to reduce his alcohol and tobacco use, and his depression, anxiety and anger lessened. However, he continued to suffer from moderate levels of all of these problems. After several months, the couple agreed to a more comprehensive treatment that included, for both of them, the following formulation, twice daily: NAC (600 mg), magnesium sulfate (75 mg), ginkgo biloba (120 mg), and glutamic acid (50 mg) as well as recommended vitamins and minerals. In addition, since the wife was not on a serotonin enhancer, she was also given 5HTP (5 mg), once daily. During this time, the husband continued to take the naltrexone, which was increased to 125 micrograms daily, and escitalopram as well as NSAIDs, as needed for break-through aches and pains. (The ULDN dose was increased based on the relative benefits of this VLDN dosing range discovered in other case studies.) In addition, they both maintain a regular intake of caffeine through daily coffee consumption.

Remarkably, within a week of the onset of this formulation, both partners were able to completely abstain from alcohol and the husband was able to stop smoking cigarettes, though he has continued to use marijuana regularly. The wife has continued to smoke cigarettes, though cut her consumption in about half. Both reported a rapid decline in symptoms of depression and anxiety. Most dramatically, the husband's anger literally disappeared, and he became calm and reasonable for the first time in years. Their moods lifted and their marital satisfaction improved significantly. A variety of fairly serious aches and pains were greatly reduced, and their sense of health and well-being improved significantly. Their sex life returned for the first time in several years, and has been very satisfying. This treatment has continued over the past six months, and these improvements have been maintained consistently throughout this time. This case study is consistent with the clear therapeutic benefits observed in all validation trials, supporting the critical importance of combining one or more Receptor Switchers with one or more Endorphin Enhancers and one or more Synergistic Enhancers, for the treatment of a wide variety of Distress Dysfunction symptoms, conditions, and disorders.

### Example 10

To assess the therapeutic benefit of the novel cotreatment formulations of the invention with Attention-Deficit Disorders, three ongoing case studies are being conducted, one focused on the use of stimulants and the other based on “nutriceuticals.”

A 13-year old male with severe uncontrolled ADHD has had serious problems with his inappropriate behaviors, emotions, and attitudes, both at school and at home, for more than 8 years. He has been taking methylphenidate (5 mg, twice daily) for the past 6 months, with only modest improvements. The medication also produced mild restlessness, sleep difficulties, and reduced appetite. NAC (600 mg, twice daily) was added to the ongoing medication. Within 3 days, observable differences were reported by both parents and teachers. The young man was able to sit quietly for longer periods of time and was clearly more cooperative and socially appropriate. Reduced side effects from the methylphenidate were also reported. Increased improvement has been observed over the past month, and the cotreatment formulation is being continued.

A 10-year old male with moderate ADHD has had difficulties paying attention at school and cooperating at home. He has never been given prescription medications for the problem, and behavioral interventions have not been successful. The child was initially given acetyl-L-carinitine (500 mg, twice daily) for a period of one week with no observable benefits. Then, NAC (600 mg, twice daily), magnesium sulfate (500 mg, twice daily), and ginkgo biloba (120 mg, once daily) were added to the ALC. Within 3 days, school and home reports indicated a significant improvement in attitude, behavior, and attention. No side effects have been observed, and an ongoing mild problem with constipation has been improved. Progress has been steadily improving, using this novel cotreatment formulation during this 3 week trial, which is ongoing.

A 24 year old male with severe ADHD has had problems in all aspects of his life due to this disorder. Despite above average intelligence, he was unable to complete high school. After dropping out of school, he was able to earn a GED. Over time, he was unable to hold regular employment. He has taken a variety of drugs for ADHD with almost no benefit, including methylphenidate. Recently, the man was given methylphenidate (5 mg), again with almost no benefit, and several undesirable side-effects, including insomnia, anorexia, and agitation. The addition of VLDN (125 micrograms) resulted in a remarkable improvement, within the first two days, in his ability to maintain attention and concentration as well as task motivation, with a significant reduction in side effects. He will continue this co-treatment formulation, given its obvious benefits.

These case studies demonstrate the relative safety and therapeutic benefits of our novel cotreatment formulations for attentional and behavioral problems, such as ADHD. Evidence suggests that conventional stimulant treatment can be significantly improved with the addition of a Receptor Switcher, such as VLDN and NAC. In addition, a pure “nutraceutical” approach, using a combination of Receptor Switchers, such as NAC and magnesium sulfate, with Endorphin and Synergistic Enhancers, such as ginkgo biloba and ALC, has potential, at least for moderate levels of ADHD.

### Example 11

In search of a topical cotreatment formulation for localized pain, loperamide was chosen since it functions as an Exogenous Opioid, but does not enter the CNS and, therefore, has minimal side effects and is available without a prescription. The cotreatment principles were used to develop a novel formulation of loperamide with MSM. A lotion was created by combining a penetrating skin cream with powdered loperamide (10-2 mg tablets) and MSM (2 g).

Four subjects (2 men, 2 women, ages 42-72) with moderate-to-severe chronic arthritic joint pain were given a supply of the formulated lotion. They were instructed to use the lotion on the painful parts of their body, up to 4 times daily, over a period of one week. All subjects reported at least a moderate improvement in joint pain that they had only experienced using prescription oral medications, which typically produced undesirable side effects. No reports of side effects were reported by the subjects. After one week, a menthol gel was added to the lotion, and 3 of the subjects reported additional relief from pain.

This application of the novel cotreatment principles of the invention has significant commercial value since it represents a potential line of safe over-the-counter products for the treatment of localized pain, which is a critical need for millions of chronic pain sufferers.

### Example 12

Given the success of the cotreatment formulations for pain that include NSAIDs, such as white willow bark, several cases studies have been initiated with pain patients who have already been using celecoxib. This prescription medication is widely used for chronic pain and inflammation, including arthritic conditions, but is not always effective and has certain undesirable side effects.

Two women (ages 56 and 72) with years of chronic arthritic pain have been taking celecoxib (200 mg daily) for over 3 years, with greatly reduced benefits over time. For one week, one woman was given ULDN (5 microgram daily), and the other was given MSM (1000 mg), in addition to ongoing celecoxib. The Receptor Switcher was reversed between the women during the second week. Both women experienced a remarkable increase in overall pain relief by the second day of this novel cotreatment, which continued during the 2 weeks. Both women were switched to VLDN (125 microgram) and celecoxib (200 mg) daily with even greater pain relief during this ongoing trial, supporting the growing evidence for the efficacy of VLDN as a Receptor Switcher.

These case studies provide further evidence for the potency of our novel non-narcotic, non-steroidal cotreatment formulations for chronic pain. The addition of a Receptor Switcher appears to not only reverse the tolerance that develops over time with celecoxib, and other analgesics, but evidence suggests that pain relief, and other therapeutic benefits, are greater with our cotreatment formulations than were experienced using the analgesic alone.

### Example 13

Based upon the discoveries of the present invention as well as previous case studies and trials, four nutraceutical formulations were developed to reduce and prevent Distress Dysfunction. These formulations are detailed below with ingredients and dose, which can be administered either once or twice daily:

A. Basic Formulation Focused on Balancing Endogenous Opioid System:

| | | |
|---|---|---|
| MSM | 600 | mg |
| NAC | 600 | mg |
| Guarana | 200 | mg |
| Ginkgo Biloba | 120 | mg |
| P5P | 20 | mg |
| Folic Acid | 0.5 | mg |
| Vitamin B-12 | 0.5 | mg |

B. Formulation Focused on Balancing and Enhancing Endogenous Opioid System:

| | | |
|---|---|---|
| MSM | 600 | mg |
| NAC | 600 | mg |
| White Willow Bark | 400 | mg |
| Guarana | 200 | mg |
| Ginkgo Biloba | 120 | mg |
| P5P | 20 | mg |
| Folic Acid | 0.5 | mg |
| Vitamin B-12 | 0.5 | mg |

C. Formulation Focused on Balancing and Enhancing Endogenous Opioid and Other Stress-Related Neurotransmitter Systems:

| | | |
|---|---|---|
| MSM | 600 | mg |
| NAC | 600 | mg |
| SAMe | 200 | mg |
| PharmaGABA | 200 | mg |
| Guarana | 200 | mg |
| Ginkgo Biloba | 120 | mg |
| 5HTP | 50 | mg |
| P5P | 20 | mg |
| Folic Acid | 0.5 | mg |
| Vitamin B-12 | 0.5 | mg |

D. Formulation Focused on Balancing and Enhancing Endogenous Opioid System and Reducing Inflammation:

| | | |
|---|---|---|
| MSM | 600 | mg |
| NAC | 600 | mg |
| Myhhr Gum | 500 | mg |
| Boswellia | 250 | mg |
| Magnesium Sulfate | 250 | mg |
| Guarana | 200 | mg |
| Ginkgo Biloba | 120 | mg |
| P5P | 20 | mg |
| Folic Acid | 0.5 | mg |
| Vitamin B-12 | 0.5 | mg |

An extensive set of case studies is underway using these nutraceutical formulations. Formulation A is primarily being used with patients suffering from mild-to-moderate emotional and physical distress. Formulations B and D are primarily being used with patients suffering from moderate-to-severe distressing and nociceptive pain. Formulation C is primarily being used with patients suffering from moderate-to-severe anxiety, depression, addictions, eating disorders, and anger. Results from these case studies will be published at the completion of the study. Preliminary findings suggest that these nutraceutical formulations are more effective than conventional treatment as well as earlier versions of these endorphinergic formulations.

### Example 14

The purpose of this example was to evaluate the effect on undesirable side effects of caffeine (CAF) consumption when at least one receptor switcher, such as N-Acetyl Cysteine (NAC), is co-administered to a person, and to determine if dosing amounts and timing affected the results.

Table 6 below presents the results of a study based on data collected from 67 female and 54 male subjects, ages 14-89 (case studies, pain and stress trials), across all dosing levels and administration schedules. All subjects were administered three or more NAC-CAF dose ratios over time. NAC and CAF were administered concurrently. The “side-effects” correlates with the percent of subjects who reported increased emotional and/or physical distress. The “well-being” column correlates with the percent of subjects who reported enhanced well being, calm, and/or comfort.

TABLE 6
SIDE-EFFECTS AND WELL-BEING AS A
FUNCTION OF NAC-CAFFEINE RATIOS

| NAC-CAF RATIO | SIDE-EFFECTS (%) | WELL-BEING (%) |
|---|---|---|
| 0:1 | 39 | 24 |
| 1:0 | 7 | 19 |
| 1:4 | 33 | 35 |
| 1:3 | 32 | 39 |
| 1:2 | 29 | 42 |
| 1:1 | 14 | 76 |
| 2:1 | 12 | 82 |
| 3:1 | 4 | 95 |
| 4:1 | 5 | 90 |
| 5:1 | 6 | 88 |

The results from this study suggest that to maximally reduce caffeine side effects and increase caffeine's benefits, that three new parameters were required: (1) specific NAC/caffeine ratio when caffeine-containing compounds are consumed; (2) specific minimum and maximum daily consumption amounts; and (3) specific 24 hours consumption schedule. By carefully following these novel parameters, the typical side effects experienced when consuming caffeine, including emotional and physical distress, anxiety and fears, agitation and restlessness, pain sensitivity as well as tolerance, dependence, and addiction to caffeine, were dramatically reduced. Furthermore, these novel parameters created combinations of NAC and caffeine that provided greater therapeutic benefits than either agent alone, including enhanced well being, calming energy, mental clarity, and bodily comfort.

The study revealed a very surprising and unexpected benefit of co-administering a Receptor Switcher with caffeine. Not only were the side effects of caffeine eliminated, but also the subjects experienced a therapeutic benefit from the combination of agents that was not produced by either agent alone. These benefits included a reduction in prior emotional and physical distress, anxiety, anger, cravings, and an increase in calm, comfort, and well-being. To be clear, these therapeutic benefits were not just the elimination of side effects of caffeine, but were improvements experienced regarding pre-existing emotional and physical distress.

The data analysis using reports from a total of 121 subjects (above) found that caffeine consumption at all levels, when taken alone, led to at least some degree of side effect report from nearly 40% of all participants, including emotional and/or physical distress. The primary benefit reported by most subjects taking caffeine alone was increased energy. These patterns are typical of those reported in prior research. However, when NAC was added, it was found that a marked decrease in noxious side effects and increase in overall well being (in addition to energy), were reported by most subjects, regardless of the amount of NAC. In other words, the co-administration of NAC, at any dose, reduced typical side effects of caffeine, and improved therapeutic benefit.

Moreover, the data revealed a remarkable and unexpected pattern, which indicated the maximal reduction in side effects and increase in benefits depended on the ratio of NAC and caffeine, as well as dosing ranges and schedules. Specifically, it was discovered that to maximally reduce side effects typically experienced with caffeine, a minimum of approximately an equal dose of NAC was indicated. Below this 1:1 level of NAC and caffeine, typical side effects of caffeine were reduced, but not nearly as effectively as ratios of NAC to caffeine of 1:1 or above. Furthermore, it was discovered that the maximal reduction of side effects was general seen when the ratio of NAC to caffeine was approximately 3: about 1. Ratios above this did not appear to significantly improve caffeine side effect reduction. Therefore, regardless of the source of caffeine, it was unexpectedly discovered that the ratio of NAC to caffeine that maximally reduced caffeine side effects was approximately in the about 1: about 1 to about 3: about 1 range. Increasing NAC above this ratio maintained this reduction side effects, but was not necessary to produce this safety profile

A second discovery stemming from analysis of the data was that the therapeutic benefits of adding NAC to caffeine were also maximized in this dosing range. Specifically, the combination of NAC and caffeine, approximately between about 1: about 1 to about 3: about 1 dosing ratio, most effectively eliminated caffeine's side effects, including emotional and physical distress, fears and anxiety, agitation and restlessness, irritability and anger, and pain sensitivity, and produced a sense of well being, calming energy, bodily comfort, and mental clarity instead. Therefore, the remarkable discoveries revealed a novel method to add specific ratios of NAC to caffeine to convert caffeine's anxiolytic characteristic to anxiety relief.

Third, while NAC has remarkably few side effects of its own, it was found that daily doses higher than 2400 mg tend to produce certain GI symptoms for some individuals. Therefore, the findings suggest that maximal caffeine side effect reduction and benefit enhancement can be achieved with an upper limit of about 2400 mg of NAC combined with about 800 mg of caffeine (about 3: about 1 ratio), consumed over the course of about 24 hours. Doses of caffeine over about 800 mg, even when combined with up to about 2400 mg of NAC, tended to produce a return of certain caffeine side effects, since the ratio of the two agents was lower than the ideal about 3: about 1 ratio. Nevertheless, the findings make it clear that caffeine, at all doses, should be combined with at least one Receptor Switcher, such as NAC, to maintain relative safety and effectiveness.

Fourth, the data analysis revealed that the receptor switcher such as NAC should be consumed at approximately the same time as the caffeine; maximal caffeine side effect reduction and benefit enhancement were seen when NAC and caffeine were consumed simultaneously. When caffeine was administered more than 30′ prior to NAC, side effects were reported. On the other hand, when NAC was administered up to four hours prior to the caffeine, side effects were minimized and benefits maximized.

It will be apparent to those skilled in the art that various modifications and variations can be made in the methods and compositions of the present invention without departing from the scope of the invention. Thus, it is intended that the present invention cover the modifications and variations of this invention provided they come within the scope of the appended claims and their equivalents.

### APPENDIX

### Bimodal Opioid Modulation of Pain and Hedonic Tone¹

¹ The attached figures (with accompanying notes) describe what is believed are the highly complex, subtle and interrelated biochemical, biological and physiological mechanisms underlying the surprising, remarkable, novel and synergistic benefits of the co-treatment formulations set forth in this patent application. The surprising, remarkable, novel and synergistic methods and combinations set forth in the patent application accurately describe the efficacy and utility of these methods and combinations to restore healthy functioning in humans and treat the conditions and disorders in humans as identified and described in this patent application.

### Healthy Homeostatic Balance

Normal homeostasis maintains an adaptive balance between the Excitatory and Inhibitory Modes in the Bimodally-Acting Opioid Receptors. In the absence of injury or stress, Opioid Receptors are generally in the Inhibitory Mode. Normal levels of Endogenous Opioids, (i.e., endorphins), are homeostatically maintained, producing a generally positive Hedonic Tone, including a sense of calm and well being

### Normal Acute Pain

Acute injury or stress triggers Acute Reflexive Pain Signals mediated by non-opioid systems, leading to the adaptive reflexive experience of immediate pain and distress. Simultaneously, acute injury or stress set Opioid Receptors in the Excitatory Mode and Endogenous Opioids (i.e., endorphins) are released. The Endogenous Opioids bind with the Opioid Receptors, triggering excitatory signaling. Through Gₛ, excitatory signals enhance the release of cAMP, which by increasing Protein Kinase A (which increases Ca² conductance and decreases K⁺ conductance), excites Pain-Sensory Neurons, which trigger the sensation of pain as well as increased sensitivity toward pain (hyperalgesia). The increased cAMP also enhances the release of Endogenous Opioids, maintaining the pain and distress cycle, leading to an extended adaptive response to the noxious stimuli. However, as soon as the acute danger is reduced, in part as a result of an adaptive response to pain and distress, the Opioid Receptors are switched to Inhibitory Mode. The Endogenous Opioids then trigger inhibitory signaling and, through Gₒ, (which decreases Ca²⁺ conductance and increases conductance), inhibit Pain-Sensory Neurons, which triggers reduced sensation of pain and produces analgesia. At the same time, the Opioid Receptor inhibitory signaling, through Gᵢ, inhibits cAMP, which in turn reduces Endogenous Opioids, which tunes down the entire endogenous opioid pain response system, restoring normal homeostatic balance and positive Hedonic Tone.

### Protracted Excitatory Mode

Chronic stress, injury, exogenous opioids, drugs, alcohol, and various medical and genetic factors can set Opioid Receptors in a protracted excitatory mode. In this condition, Endogenous Opioids trigger mostly excitatory signaling, which results in chronic pain and hyperalgesia. Any factor that triggers the release of Endogenous Opioids, including injury or stress and even reward states and various drugs, can potentiate pain. This protracted condition triggers homeostatic processes in related Serotonin, Dopamine, and other neurotransmitter systems, which produces a variety of signs and symptoms of emotional and physical distress. This negative Hedonic Tone state may be reflected by the experience of anxiety, irritability, depression, cravings, addictive tendencies and physical distress, including pain and gastrointestinal symptoms. Protracted opioid receptor excitatory mode conditions are a major component of a wide variety of Distress Dysfunction disorders, syndromes, and symptoms. Unfortunately, typical coping patterns, including the use of drugs and alcohol, perpetuate and exacerbate protracted excitatory signaling and its negative impact on Hedonic Tone.

### Exogenous Opioids

Exogenous Opioid analgesic drugs, such as tramadol, oxycodone, and morphine, clearly have a dramatic impact on the endogenous opioid system. Exogenous Opioids act like Endogenous Opioids, binding with Opioid Receptors, and their impact depends on the mode of the Bimodally-Acting Opioid Receptors. In a balanced system, their impact initially leads to inhibitory signaling, resulting in analgesia and even a sense of well being. However, fairly quickly, this increased inhibitory signaling results in a homeostatic balancing response that includes, through cAMP, a reduction in Endogenous Opioid levels as well as a receptor shift to the Excitatory Mode. Over time, this leads to a protracted excitatory receptor mode and diminished Endogenous Opioid levels, producing chronic pain, hyperalgesia, tolerance, dependence, and addiction as well as emotional and physical distress. These iatrogenic problems are greatly exacerbated when the endogenous opioid system is already in a protracted excitatory mode, resulting more immediately in excitatory signaling, leading to an exacerbation of pain and hyperalgesia, tolerance, as well as negative hedonic mode. Thus, while at times initially therapeutic, Exogenous Opioids can rapidly lead to the development of serious and Distress Dysfunction, even long after the Exogenous Opioids are discontinued.

### Receptor Switching Agents

Opioid Receptor Switchers, including ultra-low-dose and very-low-dose opioid antagonists, such as ultra-low-dose and very-low-dose naltrexone and naloxone, and GM1 ganglioside attenuators, such as neuraminidase inhibitors (e.g., magnesium sulfate and n-acetyl-cysteine), selectively block the Opioid Receptor Excitatory Mode. Therefore, protracted excitatory signaling is eliminated, and inhibitory receptor signaling is enhanced. As a result, when Endogenous Opioids (or Exogenous Opioids) bind with the Opioid Receptor, the result is increased inhibitory signaling, producing analgesia and a sense of well being. These agents have the potential to reverse both acute and protracted excitatory mode imbalances, helping to restore normal homeostatic functioning. However, since protracted excitatory conditions lead to diminished Endogenous Opioids, Receptor Switchers alone may be insufficient to produce analgesia and a sense of well being. Therefore, Receptor Switchers are most effective with co-adminstered with an agent that boosts the level of Endogenous Opioids. Alternatively, by administering a Receptor Switcher with an Exogenous Opioid, excitatory signaling is minimized, resulting in enhanced analgesia as well as a dramatic reduction in protracted excitatory mode conditions, reducing and/or eliminating many of the noxious effects of Exogenous Opioids, including tolerance, dependence, addiction, and other side effects.

### Cyclic AMP Enhancing Agents

Cyclic AMP Enhancers, particularly specific cAMP PDE-4 inhibitors, such as roflumilast, as well as non-specific cAMP PDE Inhibitors, such as theophylline and caffeine, enhance the release of cAMP, which, in turn, enhances the release of Endogenous Opioids (i.e., endorphins). In addition to cAMP PDE inhibitors, less potent cAMP Enhancers include excitatory amino acids, such as glutamic acid. When cAMP Enhancers are administered alone, the resulting increase in cAMP can directly trigger an increase in pain and hyperalgesia. Furthermore, excitatory signaling is likely if Opioid Receptors are set in the protracted excitatory mode and/or if injury or stress is present, resulting in a further increase in pain and hyperalgesia as well as emotional and physical distress. This mechanism explains the typical side effects seen with these agents. However, when co-administered with an agent that switches opioid receptors from an excitatory state to an inhibitory state, Receptor Switchers, the increase in Endogenous Opioids produced by cAMP PDE inhibitors leads to enhanced inhibitory signaling, resulting in analgesia and positive hedonic tone. Therefore, combining a Receptor Switcher and a cAMP Enhancer creates a remarkable non-opioid pharmaceutical formulation for the treatment of a wide variety of Distress Dysfunctions. Moreover, by adding a Receptor Balancer to cAMP PDE inhibitors, such as roflumilast and theophylline, enhanced formulations for the treatment of COPD and asthma are discovered with dramatically reduced side effects and increased pain relief and positive hedonic tone.

### Synergistic Enhancing Agents

A variety of agents have a synergistic effect with the endogenous opioid system through the Gᵢ-mediated metabolic processes that trigger the inhibition of pain-sensory neurons. There is evidence to suggest that higher levels of Gi that are produced by enhanced inhibitory signaling potentiate the pain-relieving effects of non-opioid analgesics, such as NSAIDs and acetaminophen. Therefore, there is a synergistic potentiation produced by the combination of non-opioid analgesics plus Receptor Switchers, such as ultra-low-dose naltrexone and neuraminidase inhibitors, creating a new generation of enhanced non-opioid analgesics. Similarly, synergistic potentiation occurs with serotonin reuptake inhibitors (SSRIs), suggesting increased pain relief as well as calm and well being is produced by the combination of SSRIs and Receptor Switchers. Therefore, a new generation of enhanced SSRIs for depression and anxiety are created by this discovery. In addition to SSRIs, inhibitory serontonergic and adrenergic agents can function as Synergistic Enhancers. Specific amino acids that enhance release of serotonin and dopamine, such as tryptophan and 5HTP, can also act as Synergistic Enhancers in cotreatment formulations. Finally, there is evidence to suggest that ultra-low-dose naltrexone (ULDN) has a synergistic effect through this Gᵢ metabolic process in addition to its function as a Receptor Switcher, making ULDN a particularly powerful agent in all cotreatment formulations.

### Endogenous Opioid Reuptake Inhibitors

Agents, such as DLPA, both enhance the release of Endogenous Opioids as well as block the enzymes that reuptake them, providing an enhanced level of Endogenous Opioids (i.e., endorphins) for longer periods of time. When administered alone, these agents have the potential for increased inhibitory signaling, but may also produce excitatory signaling when injuries or stress are present, as well as when the receptors are set in an excitatory mode. However, when co-administered with agents that switch the receptors from an excitatory to inhibitory mode, Receptor Switchers, these agents are more likely to trigger inhibitory signaling, leading to enhanced and prolonged analgesia and well being. Therefore, DLPA is an excellent agent to complement all cotreatment formulations.

### Clinical Implications

This novel understanding of the Bimodal Opioid Modulation of Pain and Hedonic Tone leads directly to new generation pharmaceutical formulations that are remarkably safe and effective for the treatment of a wide variety of Distress Dysfunctions, including chronic pain, addiction, anxiety, depression, anger, eating disorders, IBS, and other emotional and physical distress disorders. The foundation of this discovery is the power of Receptor Switchers, especially ultra-low-dose naltrexone, in blocking acute and protracted excitatory signaling. Therefore, co-administration of Receptor Switchers with cAMP Enhancing Agents is an excellent formulation for restoring healthy homeostatic balance to the endogenous opioid system, using the body's endorphins to reduce pain as well as emotional and physical distress, restoring positive hedonic tone. In this homeostatic condition, acute reflexive pain is experienced, through non-opioid systems, in response to injury or stress, but quickly is reduced when endorphins trigger inhibitory signaling. The addition of Endorphin Enhancers, such as DLPA, can enhance and prolong these therapeutic benefits. While opioid and non-opioid analgesics can potentiate these therapeutic effects when used in co-treatment with Receptor Switchers (and cAMP and Endorphin Enhancers), the evidence suggests that they can produce serious dysfunctional imbalances in the endogenous opioid system when used alone. Therefore, it is critical to co-administer Receptor Switchers whenever using opioid and non-opioid analgesic drugs to maximize their analgesic potency and to reduce noxious side effects including tolerance and dependence produced by protracted excitatory signaling. Finally, a new generation of enhanced anti-depressant and anti-anxiety medications is created by these discoveries by combining Receptor Switchers with SSRIs, which are remarkably effective in creating positive hedonic tone, including calm, well being, and pain relief.

What is claimed is:

1. A method of reducing, minimizing, or eliminating side effects associated with caffeine consumption comprising:
(a) administering caffeine to a subject; and
(b) administering at least one Receptor Switcher to the subject.

2. The method of claim 1, wherein the side effects associated with caffeine consumption are selected from the group consisting of increased stress and anxiety, agitation, restlessness, insomnia, irritability and anger, cravings, stomach pain (tenderness, bloating), constipation, unusual weakness, seizure (convulsions), twitching or uncontrolled muscle movements, fever, fast or slow heart rate, loss of appetite, anxiety, restlessness, depression, aggravation of premenstrual syndrome (PMS), fibrocystic breast disease, psychiatric side effects (confusion and psychotic symptoms), increased blood pressure, decrease in insulin sensitivity, hypoglycemia, and hyperglycemia.

3. The method of claim 1, wherein more than one receptor switcher is administered to the subject.

4. The method of claim 1, wherein the daily consumption of caffeine is selected from the group consisting of less than or equal to about 1500 mg, less than or equal to about 1400 mg, less than or equal to about 1300 mg, less than or equal to about 1200 mg, less than or equal to about 1100 mg, less than or equal to about 1000 mg, less than or equal to about 900 mg, less than or equal to about 800 mg, less than or equal to about 700 mg, with 600 mg, less than or equal to about 500 mg, less than or equal to about 400 mg, less than or equal to about 300 mg, less than or equal to about 200 mg, less than or equal to about 190, less than or equal to about 180, less than or equal to about 170, less than or equal to about 160, less than or equal to about 150, less than or equal to about 140, less than or equal to about 130, less than or equal to about 120, less than or equal to about 110, less than or equal to about 100, less than or equal to about 90, less than or equal to about 80, less than or equal to about 70, less than or equal to about 60, less than or equal to about 50, less than or equal to about 40, less than or equal to about 30, less than or equal to about 20, less than or equal to about 10, less than or equal to about 5, or less than or equal to about 1 mg of caffeine.

5. The method of claim 1, wherein at least one receptor switcher is administered simultaneously as the caffeine.

6. The method of claim 1, wherein at least one receptor switcher is administered sequentially with the caffeine.

7. The method of claim 1, wherein at least one receptor switcher is administered before the caffeine.

8. The method of claim 10, wherein at least one receptor switcher is administered about 1, about 2, about 3, about 4, about 5, about 6, about 7, about 8, about 9, about 10, about 15, about 30, about 45, about 60, about 75, about 90, about 105, about 120, about 135, about 150, about 165, about 180, or about 200 min. before the caffeine.

9. The method of claim 1, wherein at least one receptor switcher is administered at any time within 24 hours prior to caffeine consumption.

10. The method of claim 1, wherein at least one receptor switcher is administered at any time within 24 hours following caffeine consumption.

11. The method of claim 1, wherein the Receptor Switcher is selected from the group consisting of agents that selectively block and/or inhibit opioid receptor excitatory signaling, ultra-low-dose, very-low-dose and low dose opioid antagonists, ultra-low-dose naltrexone, very-low-dose naltrexone, ultra-low-dose naloxone, very-low-dose naloxone, diprenorphine, nalmefene, norbinaltorphimine, agents that inhibit synthesis or activity of GM1 ganglioside, neuraminidase inhibitors, agents that increase sulfates in the body, methylsulfonylmethane (MSM), magnesium sulfate, sodium sulfate, chondroitin sulfate, n-acetyl-cysteine (NAC), zanamivir, laninamivir, peramivir, oseltamivir, scutellaria, and 5,7,4′-trihydroxy-8-methoxyflavone.

12. The method of claim 1, wherein the receptor switcher is n-acetyl-cysteine (NAC).

13. The method of claim 1, wherein NAC and caffeine are administered in a ratio selected from the group consisting of about 1 to about 2, about 1 to about 1.9, about 1 to about 1.8, about 1 to about 1.7, about 1 to about 1.6, about 1 to about 1.5, about 1 to about 1.4, about 1 to about 1.3, about 1 to about 1.2, about 1 to about 1.1, about 1 to about 1, about 1.1 to about 1, about 1.2 to about 1, about 1.3 to about 1, about 1.4 to about 1, about 1.5 to about 1, about 1.6 to about 1, about 1.7 to about 1, about 1.8 to about 1, about 1.9 to about 1, about 2 to about 1, about 2.1 to about 1, about 2.2 to about 1, about 2.3 to about 1, about 2.4 to about 1, about 2.5 to about 1, about 2.6 to about 1, about 2.7 to about 1, about 2.8 to about 1, about 2.9 to about 1, about 3 to about 1, about 3.1 to about 1, about 3.2 to about 1, about 3.3 to about 1, about 3.4 to about 1, about 3.5 to about 1, about 3.6 to about 1, about 3.7 to about 1, about 3.8 to about 1, about 3.9 to about 1, about 4 to about 1, and above 4 to about 1.

14. The method of claim 4, wherein NAC and caffeine are administered in a ratio of about or above 1: about 1.

15. The method of claim 1, wherein NAC is administered at a daily dose selected from the group consisting of less than or equal to about 3000 mg, less than or equal to about 2900 mg, less than or equal to about 2800 mg, less than or equal to about 2700 mg, less than or equal to about 2600 mg, less than or equal to about 2500 mg, less than or equal to about 2400 mg, less than or equal to about 2300 mg, less than or equal to about 2200 mg, less than or equal to about 2100 mg, less than or equal to about 2000 mg, less than or equal to about 1900 mg, less than or equal to about 1800 mg, less than or equal to about 1700 mg, less than or equal to about 1600 mg, less than or equal to about 1500 mg, less than or equal to about 1400 mg, less than or equal to about 1300 mg, less than or equal to about 1200 mg, less than or equal to about 1100 mg, less than or equal to about 1000 mg, less than or equal to about 900 mg, less than or equal to about 800 mg, less than or equal to about 700 mg, less than or equal to about 600 mg, less than or equal to about 500 mg, less than or equal to about 400 mg, less than or equal to about 300 mg, less than or equal to about 200 mg, less than or equal to about 100 mg/day, less than or equal to about 50 mg, or less than or equal to about 25 mg/day,

16. The method of claim 1, wherein the Receptor Switcher:
(a) is an ultra-low-dose, very-low-dose, or low-dose opioid antagonist; or
(b) is ultra-low-dose, very-low-dose, or low-dose naltrexone or naloxone.

17. The method of claim 15, wherein:
(a) naltrexone is administered in the ultra-low-dose amount of about 125 micrograms or less;
(b) naltrexone is administered in the very-low-dose range of about 125-about 500 micrograms;
(c) naltrexone is administered in the low-dose range of about 500-about 5000 micrograms;
(d) naloxone is administered at an ultra-low-dose of about 0.15 nanagrams·kg⁻¹·h⁻¹ to 0.25 μg·kg⁻¹·h⁻¹;
(e) naloxone is administered at about 400 micrograms naloxone in 1000 ml crystalloid given in 24 h to a patient weighing 70 kg;
(f) naloxone is administered at a low-dose of about 0.25 μg·kg⁻¹·h⁻¹ to 1.0 μg·kg⁻¹·⁻¹; or
(g) naloxone is administered at as dose of about 1 μg·kg⁻¹·h⁻¹ to about 5 μg·kg⁻¹·h⁻¹.

18. The method of claim 1, wherein:
(a) the mode of administration is selected from the group consisting of oral, pulmonary, nasal, sublingual, parenteral, and transdermal;
(b) the pharmaceutical formulation is delivered in a pharmaceutically-acceptable carrier that is rapid release, immediate-release, slow-release, delayed-released, controlled release, a combination of immediate release and controlled release, a nano-encapsulation formulations, and an abuse and/or tamper-resistant delivery system; or
(c) any combination thereof.

19. A composition comprising:
(a) caffeine: and
(b) at least one Receptor Switcher selected from the group consisting of agents that selectively block and/or inhibit opioid receptor excitatory signaling, ultra-low-dose, very-low-dose and low dose opioid antagonists, ultra-low-dose naltrexone, very-low-dose naltrexone, ultra-low-dose naloxone, very-low-dose naloxone, diprenorphine, nalmefene, norbinaltorphimine, agents that inhibit synthesis or activity of GM1 ganglioside, neuraminidase inhibitors, agents that increase sulfates in the body, methylsulfonylmethane (MSM), magnesium sulfate, sodium sulfate, chondroitin sulfate, n-acetyl-cysteine (NAC), zanamivir, laninamivir, peramivir, oseltamivir, scutellaria, and 5, 7,4′-trihydroxy-8-methoxyflavone.

20. The composition of claim 18 formulated into a dosage form selected from the group consisting of rapid release, immediate-release, slow-release, delayed-released, controlled release, a combination of controlled release and immediate release, nano-encapsulation formulations, and an abuse-resistant delivery system.
